SEQUENCE LISTING (37 sequences)

SEQ ID NO: 1 - protein, 4 aa, Artificial Sequence
  Description of Artificial Sequence Synthetic peptide
KYVI

SEQ ID NO: 2 - protein, 4 aa, Artificial Sequence
  Description of Artificial Sequence Synthetic peptide
YPVI

SEQ ID NO: 3 - protein, 5 aa, Artificial Sequence
  Description of Artificial Sequence Synthetic peptide
EEPPI

SEQ ID NO: 4 - protein, 5 aa, Artificial Sequence
  Description of Artificial Sequence Synthetic peptide
DDGGI

SEQ ID NO: 5 - protein, 5 aa, Artificial Sequence
  Description of Artificial Sequence Synthetic peptide
DDLLI

SEQ ID NO: 6 - protein, 5 aa, Artificial Sequence
  Description of Artificial Sequence Synthetic peptide
DDLLI

SEQ ID NO: 7 - protein, 5 aa, Artificial Sequence
  Description of Artificial Sequence Synthetic peptide
DDPPI

SEQ ID NO: 8 - protein, 5 aa, Artificial Sequence
  Description of Artificial Sequence Synthetic peptide
EEGGF

SEQ ID NO: 9 - protein, 5 aa, Artificial Sequence
  Description of Artificial Sequence Synthetic peptide
EELLL

SEQ ID NO: 10 - protein, 5 aa, Artificial Sequence
  Description of Artificial Sequence Synthetic peptide
EEFFL

SEQ ID NO: 11 - protein, 5 aa, Artificial Sequence
  Description of Artificial Sequence Synthetic peptide
EEFPI

SEQ ID NO: 12 - protein, 5 aa, Artificial Sequence
  Description of Artificial Sequence Synthetic peptide
EEPPL

SEQ ID NO: 13 - protein, 5 aa, Artificial Sequence
  Description of Artificial Sequence Synthetic peptide
EEPFI

SEQ ID NO: 14 - protein, 5 aa, Artificial Sequence
  Description of Artificial Sequence Synthetic peptide
EEEEI

SEQ ID NO: 15 - protein, 5 aa, Artificial Sequence
  Description of Artificial Sequence Synthetic peptide
EEFFF

SEQ ID NO: 16 - protein, 5 aa, Artificial Sequence
  Description of Artificial Sequence Synthetic peptide
GGEEI

SEQ ID NO: 17 - protein, 5 aa, Artificial Sequence
  Description of Artificial Sequence Synthetic peptide
KKLLI

SEQ ID NO: 18 - protein, 5 aa, Artificial Sequence
  Description of Artificial Sequence Synthetic peptide
KKPPI

SEQ ID NO: 19 - protein, 5 aa, Artificial Sequence
  Description of Artificial Sequence Synthetic peptide
FFEEI

SEQ ID NO: 20 - protein, 5 aa, Artificial Sequence
  Description of Artificial Sequence Synthetic peptide
FFFFF

SEQ ID NO: 21 - protein, 5 aa, Artificial Sequence
  Description of Artificial Sequence Synthetic peptide
TTGGI

SEQ ID NO: 22 - protein, 5 aa, Artificial Sequence
  Description of Artificial Sequence Synthetic peptide
TTFFI

SEQ ID NO: 23 - protein, 5 aa, Artificial Sequence
  Description of Artificial Sequence Synthetic peptide
YYLLI

SEQ ID NO: 24 - protein, 5 aa, Artificial Sequence
  Description of Artificial Sequence Synthetic peptide
YYFFI

SEQ ID NO: 25 - protein, 5 aa, Artificial Sequence
  Description of Artificial Sequence Synthetic peptide
YYPPI

SEQ ID NO: 26 - protein, 5 aa, Artificial Sequence
  Description of Artificial Sequence Synthetic peptide
YYPFI

SEQ ID NO: 27 - protein, 5 aa, Artificial Sequence
  Description of Artificial Sequence Synthetic peptide
YYFFI

SEQ ID NO: 28 - protein, 5 aa, Artificial Sequence
  Description of Artificial Sequence Synthetic peptide
YYFFV

SEQ ID NO: 29 - protein, 5 aa, Artificial Sequence
  Description of Artificial Sequence Synthetic peptide
DDLDD

SEQ ID NO: 30 - protein, 5 aa, Artificial Sequence
  Description of Artificial Sequence Synthetic peptide
EEKEE

SEQ ID NO: 31 - protein, 5 aa, Artificial Sequence
  Description of Artificial Sequence Synthetic peptide
FFKFF

SEQ ID NO: 32 - protein, 5 aa, Artificial Sequence
  Description of Artificial Sequence Synthetic peptide
PPKPP

SEQ ID NO: 33 - protein, 5 aa, Artificial Sequence
  Description of Artificial Sequence Synthetic peptide
YYKYY

SEQ ID NO: 34 - protein, 5 aa, Artificial Sequence
  Description of Artificial Sequence Synthetic peptide
EEFFI

SEQ ID NO: 35 - protein, 4 aa, Artificial Sequence
  Description of Artificial Sequence Synthetic peptide
KKGG

SEQ ID NO: 36 - protein, 5 aa, Artificial Sequence
  Description of Artificial Sequence Synthetic peptide
GGKGG

SEQ ID NO: 37 - protein, 4 aa, Artificial Sequence
  Description of Artificial Sequence Synthetic peptide
GGKG

(19) United States
(12) Patent Application Publication
(10) Pub. No.: US 2011/0040072 A1
(43) Pub. Date: Feb. 17, 2011

(54) MONO AND DI-SUBSTITUTED OXYCODONE COMPOUNDS AND COMPOSITIONS

(75) Inventors: Travis Mickle, Coralville, IA (US); Suma Krishnan, Belvedere, CA (US); James Scott Moncrief, Christiansburg, VA (US); Christopher Lauderback, Lovettsville, VA (US); Sanjib Bera, Blacksburg, VA (US)

Correspondence Address: DARBY & DARBY P.C., P.O. BOX 770, Church Street Station, New York, NY, 10008-0770, US

(73) Assignee: SHIRE LLC, Florence, KY (US)

(21) Appl. No.: 12/722,495

(22) Filed: Mar. 11, 2010

Related U.S. Application Data
(63) Continuation of application No. 12/296,368, now abandoned.
(60) Provisional application No. 60/790,524, filed on Apr. 10, 2006.
(60) Provisional application No. 60/796,352, filed on May 1, 2006.
(60) Provisional application No. 60/849,775, filed on Oct. 6, 2006.

(51) Int. Cl. C07K 7/06; C07D 489/08; C07K 5/087

(52) U.S. Cl. 530/330; 546/44; 530/331

(57) ABSTRACT

The invention relates to pharmaceutical compounds and compositions comprised of a chemical moiety attached to oxycodone in a manner that substantially decreases the potential for overdose. When delivered at the proper dosage the pharmaceutical composition provides therapeutic activity similar to that of oxycodone and may also provide sustained release characteristics and/or reduced side-effects. Further the compounds and compositions of the invention are useful in preventing addiction and susceptibility to addiction of oxycodone.

### CROSS-REFERENCE RELATED APPLICATIONS

This application claims benefit under 35 U.S.C. §119(e) to U.S. Provisional Application No. 60/796,352 filed on May 1, 2006, claims benefit under 35 U.S.C. §119(e) to U.S. Provisional Application 60/790,524 filed on Apr. 10, 2006, and claims benefit under 35 U.S.C. §119(e) to U.S. Provisional Application 60/849,775 filed Oct. 6, 2006 each of which are hereby incorporated by reference in their entirety.

### FIELD OF INVENTION

The present invention relates to pharmaceutical compounds, compositions, and methods of using the same comprising a chemical moiety attached to oxycodone. These inventions provide a variety of beneficial effects. Some inventions result in a substantial decrease in the potential of oxycodone to cause overdose or to be abused. For instance, some inventions provide therapeutic activity similar to that of the parent oxycodone when delivered at typical dosage ranges, however, when delivered at higher doses the potential for overdose is reduced due to the limited bioavailability of oxycodone as compared to oxycodone delivered in an non-conjugated form. Alternatively or in addition, the prodrug may be designed to provide fast or slow release depending on its use for chronic versus acute pain. Additionally, the compounds and compositions of the invention may reduce side-effects associated with taking oxycodone.

### BACKGROUND

Accidental and intentional overdose with prescription and over the counter drugs is a serious health problem with thousands of fatalities occurring each year as a result. Drug overdose is a significant and growing problem. It can occur accidentally, as when a child swallows pills without understanding the consequences, or intentionally as with suicide attempts. In addition, accidental overdose due to an unusually potent batch of a street drug in illicit drug users is quite common. Emergency department reporting for a number of drugs rose substantially from 1994 to 2000. These include: amphetamines (10,118 to 18,555, up 83.4%), anticonvulsants, including carbamazepine (9,358 to 14,642, up 56.5%), muscle relaxants, including carisoprodol (12,223 to 19,001, up 55.5%), psychotherapeutic drugs, including SSRI antidepressants, tricyclic antidepressants, and other antidepressants (190,467 to 220,289, up 15.7%). Anxiolytics, sedatives, and hypnotics, including benzodiazepines (74,637 to 103,972, up 27.7%) and narcotic analgesics including codeine, hydrocodone, methadone, oxycodone, propoxyphene and others (44,518 to 99,317, up 123.1%).

Others have sought to prevent the potential harmful effects of overdose through various formulations. For example, opioids have been combined with antagonists in particular formulations designed to counteract the opioid if the formulation is disrupted before oral administration or is given parenterally. Extended release Concerta (methylphenidate) has been formulated in a paste to preclude administration by snorting or injection. Compositions have been coated with emetics in a quantity that if administered in moderation as intended no emesis occurs, however, if excessive amounts are consumed emesis is induced therefore preventing overdose. These methods, as well as conventional control release formulations, are often ineffective and circumvented.

The opioid oxycodone is an ingredient of Percodan, Percocet, Roxicet, and Tylox. It is a semisynthetic narcotic analgesic that is derived from thebaine. Available in oral formulations often in combination with aspirin, phenacetin and caffeine. Typical adult dose is 2.5-5 mg as the hydrochloride or terephthalate salt every 6 hours. Although it is typically used for the relief of moderate to moderately severe pain, it can also produce drug dependence of the morphine type. Therapeutic plasma concentration is 10-100 ng/mL and the toxic plasma concentration is greater than 200 ng/mL.

Consequently, improved methods are needed to make pharmaceutically effective oxycodone compounds, compositions and methods of using the same with reduced potential for overdose and/or resistance to manipulation while still providing necessary analgesia for various types of pain. Preferably, absorption of the composition into the brain is prevented or substantially diminished and/or delayed when delivered by routes other than oral administration.

### BRIEF DESCRIPTION OF THE FIGURES

FIG. 1 depicts the numbering scheme for oxycodone.

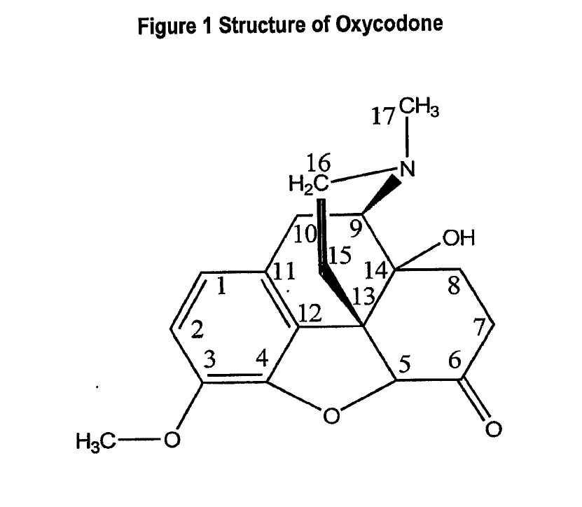

FIG. 2 depicts oxycodone conjugated at the 6 position.

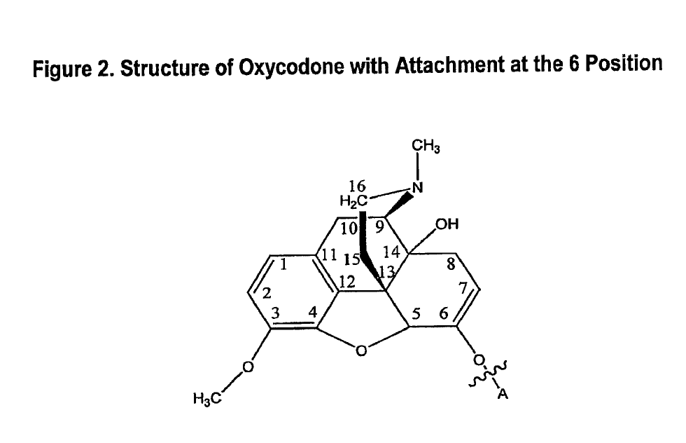

FIG. 3 depicts oxycodone conjugated at the 6 and 14 positions.

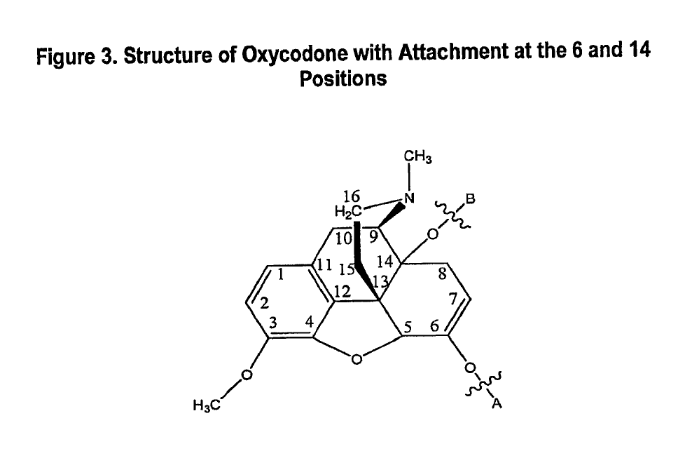

FIG. 4 depicts oxycodone conjugated at the 14 position.

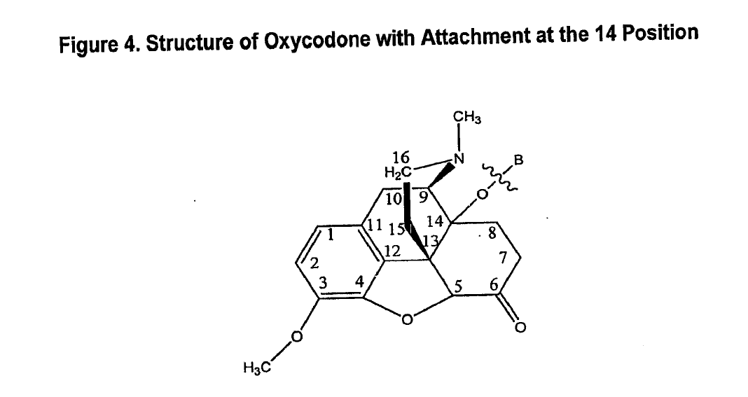

FIG. 5 depicts Oral Bioavailability of Disubstituted Peptide Oxycodone Compounds.

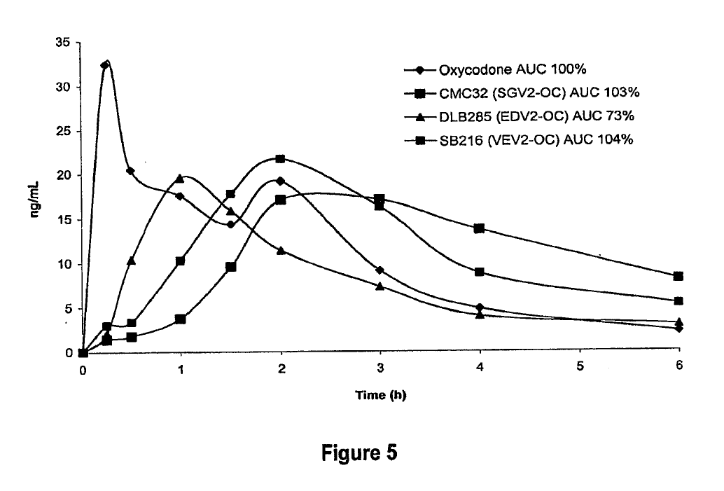

FIG. 6 depicts Oral Bioavailability of Monosubstituted Peptide Oxycodone Compounds.

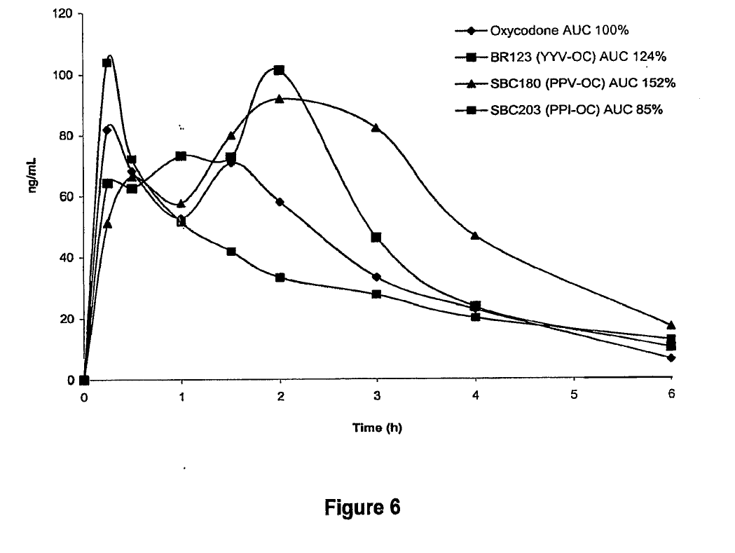

FIG. 7 depicts Oral Bioavailability of Non-Natural Single Amino Acid Oxycodone Compounds.

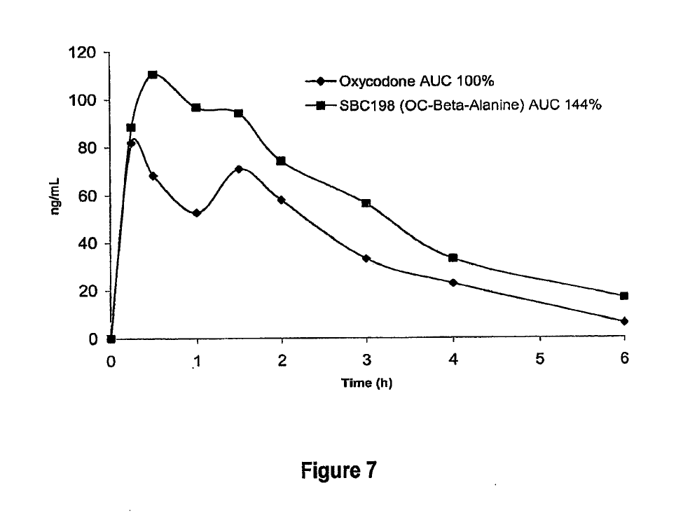

FIG. 8 depicts Intranasal Bioavailability of Disubstituted Peptide Oxycodone Compounds.

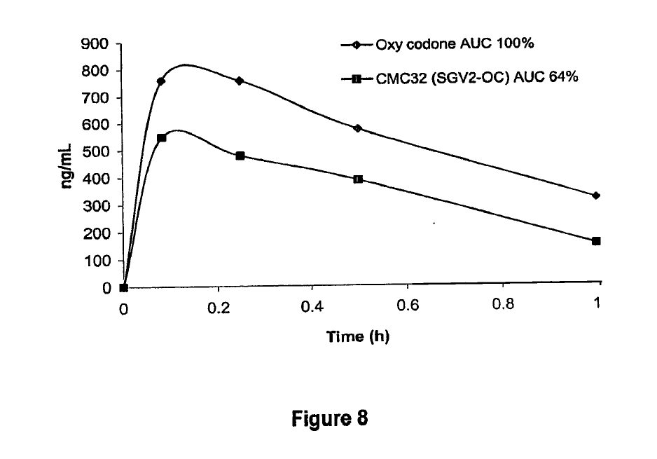

FIG. 9 depicts Intranasal Bioavailability of Disubstituted Peptide Oxycodone Compounds.

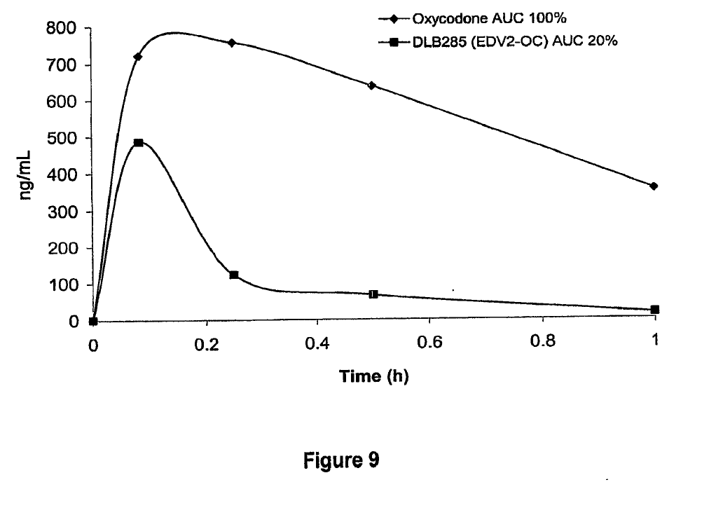

FIG. 10 depicts Intranasal Bioavailability of Disubstituted Peptide Oxycodone Compounds.

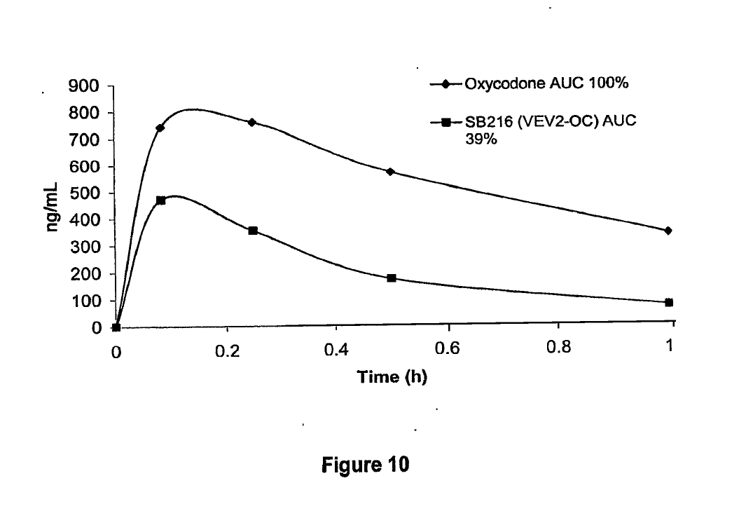

FIG. 11 depicts Intravenous Bioavailability of Disubstituted Peptide Oxycodone Compounds.

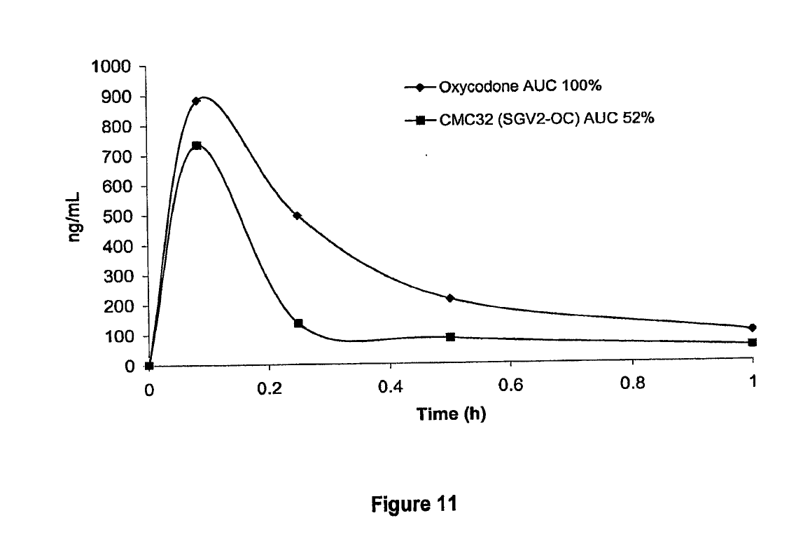

### DETAILED DESCRIPTION OF THE INVENTION

The invention relates to changing the pharmacokinetic and pharmacological properties of oxycodone through covalent modification. Covalent attachment of a chemical moiety to oxycodone may change one or more of the following: the rate of absorption, the extent of absorption, the metabolism, the distribution, and the elimination (ADME pharmacokinetic properties) of oxycodone. As such, the alteration of one or more of these characteristics may be designed to provide fast or slow release depending on its use for chronic pain versus acute pain. Additionally, alteration of one or more of these characteristics may reduce the side effects associated with taking oxycodone

One aspect of the invention includes oxycodone conjugates that when administered at a normal therapeutic dose the bioavailability (area under the time-versus-concentration curve; AUC) of oxycodone provides a pharmaceutically effective amount of oxycodone. As the dose is increased, however, the bioavailability of the covalently modified oxycodone relative to the parent oxycodone begins to decline, particularly for oral dosage forms. At suprapharmacological doses the bioavailability of the oxycodone conjugate is substantially decreased as compared to the parent oxycodone. The relative decrease in bioavailability at higher doses decreases or reduces the euphoria obtained when doses of the oxycodone conjugate are taken above those of the intended prescription. This in turn diminishes the abuse potential, whether unintended or intentionally sought.

The invention provides oxycodone prodrugs comprising oxycodone covalently bound to a chemical moiety. The oxycodone prodrugs can also be characterized as conjugates in that they possess a covalent attachment. They may also be characterized as conditionally bioreversible derivatives (“CBDs”).

In one embodiment, the oxycodone prodrug (a compound of one of the formulas described herein) may exhibit one or more of the following advantages over free oxycodone. The oxycodone prodrug may prevent overdose by exhibiting a reduced pharmacological activity when administered at higher than therapeutic doses, e.g., higher than the prescribed dose. Yet when the oxycodone prodrug is administered at therapeutic doses, the oxycodone prodrug may retain similar pharmacological activity to that achieved by administering unbound oxycodone. Also, the oxycodone prodrug may prevent abuse by exhibiting stability under conditions likely to be employed by illicit chemists attempting to release the oxycodone. The oxycodone prodrug may prevent abuse by exhibiting reduced bioavailability when it is administered via parenteral routes, particularly the intravenous (“shooting”), intranasal (“snorting”), and/or inhalation (“smoking”) routes that are often employed in illicit use. Thus, the oxycodone prodrug may reduce the euphoric effect associated with oxycodone abuse. Thus, the oxycodone prodrug may prevent and/or reduce the potential of abuse and/or overdose when the oxycodone prodrug is used in a manner inconsistent with the manufacturer's instructions, e.g., consuming the oxycodone prodrug at a higher than therapeutic dose or via a non-oral route of administration.

Preferably, the oxycodone prodrug provides a serum release curve that does not increase above oxycodone's toxicity level when administered at higher than therapeutic doses. The oxycodone prodrug may exhibit a reduced rate of oxycodone absorption and/or an increased rate of clearance compared to the free oxycodone. The oxycodone prodrug may also exhibit a steady-state serum release curve. Preferably, the oxycodone prodrug provides bioavailability but prevents Cₘₐₓ spiking or increased blood serum concentrations.

Oxycodone may be bound to one or more chemical moieties, denominated X and Z. A chemical moiety can be any moiety that decreases the pharmacological activity of oxycodone while bound to the chemical moiety as compared to unbound (free) oxycodone. The attached chemical moiety can be either naturally occurring or synthetic. In one embodiment, the invention provides an oxycodone prodrug of Formula IA or IB:

O—Xₙ—Zₘ (IA)

O—Zₘ—Xₙ (IB)

wherein O is oxycodone;
each X is independently a chemical moiety;
each Z is independently a chemical moiety that acts as an adjuvant and is different from at least one X;
n is an increment from 1 to 50, preferably 1 to 10; and
m is an increment from 0 to 50, preferably 0.
When m is 0, the oxycodone prodrug is a compound of Formula (II):

O—Xₙ (II)

wherein each X is independently a chemical moiety.

Formula (II) can also be written to designate the chemical moiety that is physically attached to the oxycodone:

O—O₁—(X)ₙ₋₁ (III)

wherein O is oxycodone; X₁ is a chemical moiety, preferably a single amino acid; each X is independently a chemical moiety that is the same as or different from X₁; and n is an increment from 1 to 50.

O is oxycodone and upon substitution with X, may have the following structures IV, V, or VI, wherein A and B represent possible attachment sites for X.

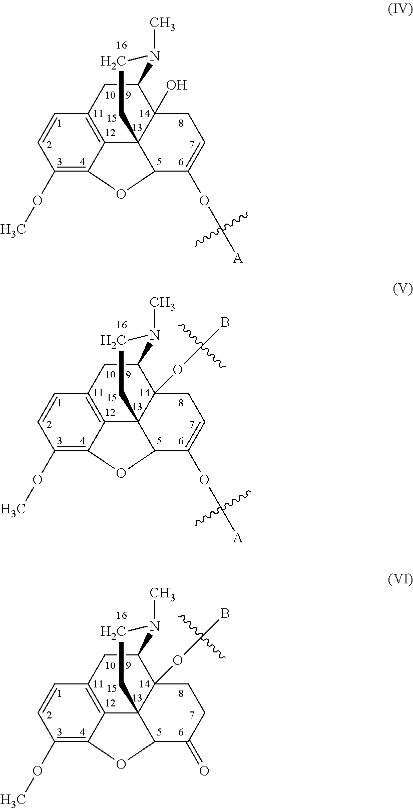

In an alternative embodiment, the 3 position and/or N position of oxycodone may be substituted with a chemical moiety with or without the presence of a linker. See U.S. Pat. No. 5,610,283 for methods of substituting opioids at these positions. Chemical moieties include, but are not limited to any of the carrier peptides listed below in Table 1.

Compounds, compositions and methods of the invention provide reduced potential for overdose, reduced potential for abuse or addiction and/or improve oxycodone's characteristics with regard to high toxicities or suboptimal release profiles. Without wishing to be limited to the below theory, we believe that in some instances overdose protection results from a natural gating mechanism at the site of hydrolysis that limits the release of oxycodone from the prodrug at greater than therapeutically prescribed amounts. Therefore, abuse resistance is provided by limiting the “rush” or “high” available from the oxycodone released by the prodrug and limiting the effectiveness of alternative routes of administration for certain chemical moieties.

The invention utilizes covalent modification of oxycodone to alter its ADME for certain delivery routes, e.g. routes other than oral, to decrease its potential for causing overdose or being abused. The oxycodone is covalently modified in a manner that decreases its pharmacological activity, as compared to the unmodified oxycodone, at doses above those considered therapeutic, e.g., at doses inconsistent with the manufacturer's instructions. When given at lower doses, such as those intended for therapy, covalently modified oxycodone retains effective pharmacological activity. The covalent modification of oxycodone may comprise the attachment of any chemical moiety through conventional chemistry. Preferably the chemical moiety is a carrier peptide.

Further, at times the invention is described as being oxycodone attached to an amino acid, a dipeptide, a tripeptide, tetrapeptide, pentapeptide, or hexapeptide to illustrate specific embodiments for the oxycodone conjugate. Preferred lengths of the conjugates and other preferred embodiments are described herein. Preferred carriers are listed in Tables 1 and 2.

Persons that abuse prescription drugs commonly seek to increase their euphoria by snorting or injecting the drugs. These routes of administration increase the rate and extent of drug absorption and provide a faster, nearly instantaneous, effect. This increases the amount of drug that reaches the central nervous system where it has its effect. In a particular embodiment of the invention the bioavailability of the covalently modified oxycodone is substantially decreased when taken by the intranasal and intravenous routes as compared to the parent oxycodone. Thus the illicit practice of snorting and shooting the drug loses its advantage, i.e., the central nervous system effects are diminished.

In another embodiment of the invention, the solubility and dissolution rate of the composition is substantially changed under physiological conditions encountered in the intestine, at mucosal surfaces, or in the bloodstream. In another embodiment the solubility and dissolution rate substantially decrease the bioavailability of the oxycodone prodrug, particularly at doses above those intended for therapy. In another embodiment the decrease in bioavailability occurs upon oral administration. In another embodiment the decrease in bioavailability occurs upon intranasal administration. In another embodiment the decrease in bioavailability occurs upon intravenous administration.

Another particular embodiment of the invention provides that when the covalently modified oxycodone is provided in oral dosage form (e.g., a tablet, capsule, caplet, liquid dispersion, etc.) it has increased resistance to manipulation. For instance, crushing of a tablet or disruption of a capsule does not substantially increase the rate and amount of oxycodone absorbed when compositions of the invention are ingested.

Another embodiment of the invention provides compositions and methods of providing analgesia comprising administering to a patient compounds or compositions of the invention. Another embodiment provides a composition or method for treating pain in a patient i.e., acute and chronic pain—it should be noted that different conjugates maybe be utilized to treat acute versus chronic pain.

Oxycodone may be attached to the carrier peptide through the C-terminus, N-terminus, or side chain of the carrier peptide. Preferably, oxycodone is attached to the C-terminus of the carrier peptide. It is preferred that aside from attachment of the carrier peptide to the oxycodone neither is further substituted or protected. In one embodiment, the chemical moiety has one or more free carboxy and/or amine terminal and/or side chain group other than the point of attachment to the oxycodone. The chemical moiety can be in such a free state, or an ester or salt thereof.

Another embodiment of the invention is a composition or method for safely delivering oxycodone comprising providing a therapeutically effective amount of said oxycodone which has been covalently bound to a chemical moiety wherein said chemical moiety reduces the rate of absorption of the oxycodone as compared to delivering the unbound oxycodone.

Another embodiment of the invention is a composition or method for reducing drug toxicity comprising providing a patient with oxycodone which has been covalently bound to a chemical moiety wherein said chemical moiety increases the rate of clearance of oxycodone when given at doses exceeding those within the therapeutic range of said oxycodone.

Another embodiment provides a composition or method of reducing drug toxicity comprising providing a patient with oxycodone which has been covalently bound to a chemical moiety wherein the chemical moiety provides a serum release curve which does not increase above the toxicity level of oxycodone when given at doses exceeding those within the therapeutic range for unbound oxycodone.

Another embodiment provides a composition that reduces or eliminates the toxic range of the Lethal Dose, 50% (LD₅₀) comprising providing a composition containing oxycodone, which has been covalently bound to a chemical moiety.

Another embodiment of the invention is a composition or method for a sustained-release oxycodone composition comprising providing oxycodone which has been covalently bound to a chemical moiety, wherein said chemical moiety provides release of oxycodone at a rate where the level of oxycodone is within the therapeutic range but below toxic levels over an extended periods of time, e.g., 8-24 hours or greater.

Another embodiment of the invention is a composition or method for reducing bioavailability or preventing a toxic release profile of oxycodone comprising oxycodone covalently bound to a chemical moiety wherein said bound oxycodone maintains a steady-state serum release curve which provides a therapeutically effective bioavailability but prevents spiking or increase blood serum concentrations compared to unbound oxycodone when given at doses exceeding those within the therapeutic range of said oxycodone.

Another embodiment of the invention is a composition or method for preventing a Cₘₐₓ spike for oxycodone while still providing a therapeutically effective bioavailability curve comprising oxycodone which has been covalently bound to a chemical moiety.

In another embodiment the compositions have substantially lower toxicity compared to unbound oxycodone. In another embodiment the compositions reduce or eliminate the possibility of overdose by oral administration. In another embodiment the compositions reduce or eliminate the possibility of overdose by intranasal administration. In another embodiment the compositions reduce or eliminate the possibility of overdose by injection.

The invention further provides compositions or methods for altering oxycodone in a manner that decreases their potential for abuse. Compositions and methods of the invention provide various ways to regulate pharmaceutical dosage through covalent attachment of oxycodone to different chemical moieties. One embodiment provides a method of preventing overdose comprising administering to an individual oxycodone which has been covalently bound to a chemical moiety.

Another embodiment of the invention is a method for reducing or preventing abuse or euphoric effect of a pharmaceutical composition, comprising providing, administering, or prescribing said composition to a human in need thereof, wherein said composition comprises a chemical moiety covalently attached to oxycodone such that the pharmacological activity of oxycodone is substantially decreased when the composition is used in a manner inconsistent with the manufacturer's instructions or in a manner that substantially increases the potential of overdose from oxycodone.

Another embodiment of the invention is a method for reducing or preventing abuse or euphoric effect of a pharmaceutical composition, comprising consuming said composition, wherein said composition comprises a chemical moiety covalently attached to oxycodone such that the pharmacological activity of oxycodone is substantially decreased when the composition is used in a manner inconsistent with the manufacturer's instructions or in a manner that substantially decreases the potential of overdose from oxycodone.

Another embodiment of the invention is any of the preceding methods wherein said pharmaceutical composition is adapted for oral administration, and wherein said oxycodone is resistant to release from said chemical moiety when the composition is administered parenterally, such as intranasally or intravenously. Preferably, said oxycodone may be released from said chemical moiety in the presence of acid and/or enzymes present in the stomach, intestinal tract, or blood serum.

Another embodiment of the invention is any of the herein described methods wherein said composition yields a therapeutic effect without substantial euphoria. Preferably, said oxycodone provides a therapeutically bioequivalent AUC when compared to oxycodone alone but does not provide a Cₘₐₓ which results in euphoria.

Another embodiment of the invention is a method for reducing or preventing abuse of a pharmaceutical composition, comprising orally administering said composition to a human in need thereof, wherein said composition comprises an amino acid or peptide covalently attached to oxycodone such that the pharmacological activity of oxycodone is substantially decreased when the composition is used in a manner inconsistent with the manufacturer's instructions.

Another embodiment is a method of preventing overdose of a pharmaceutical composition, comprising orally administering said pharmaceutical composition to a human in need thereof, wherein said composition comprises a carrier peptide covalently attached to oxycodone in a manner that substantially decreases the potential of oxycodone to result in overdose.

Another embodiment is a method for reducing or preventing the euphoric effect of a pharmaceutical composition, comprising orally administering said composition to a human in need thereof, wherein said composition comprises a carrier peptide covalently attached to oxycodone such that the pharmacological activity of oxycodone is substantially decreased when the composition is used in a manner inconsistent with the manufacturer's instructions.

For each of the recited methods of the invention the following properties may be achieved through bonding oxycodone to the chemical moiety. In one embodiment, the toxicity of the compound may be substantially lower than that of the oxycodone when delivered in its unbound state or as a salt thereof. In another embodiment, the possibility of overdose by oral administration is reduced or eliminated. In another embodiment, the possibility of overdose by intranasal administration is reduced or eliminated. In another embodiment, the possibility of overdose by injection administration is reduced or eliminated.

Another embodiment of the invention is wherein said attachment comprises an ester or carbonate bond. Another embodiment of the invention is wherein said oxycodone covalently attaches to a chemical moiety through a ketone and/or hydroxyl.

The compositions and methods of the invention provide oxycodone, which when bound to the chemical moiety provide safer and/or more effective dosages for oxycodone through improved bioavailability curves and/or safer Cₘₐₓ and/or reduce area under the curve for bioavailability, particularly for abused substances taken in doses above therapeutic levels. As a result, the compositions and methods of the invention may provide improved methods of treatment for analgesia.

Preferably, the oxycodone prodrug exhibits an oral bioavailability of oxycodone of at least about 60% AUC (area under the curve), more preferably at least about 70%, 80%, 90%, 95%, 96%, 97%, 98%, 99%, compared to unbound oxycodone. Preferably, the oxycodone prodrug exhibits a parenteral bioavailability, e.g., intranasal, bioavailability of less than about 70% AUC, more preferably less than about 50%, 30%, 20%, 15%, 10%, 5%, 4%, 3%, 2%, 1%, compared to unbound oxycodone.

In one embodiment, the oxycodone prodrug provides pharmacological parameters (AUC, Cₘₐₓ, Tₘₐₓ, Cₘᵢₙ, and/or t_{1/2}) within 80% to 125%, 80% to 120%, 85% to 125%, 90% to 110%, or increments therein of unbound oxycodone. It should be recognized that the ranges can, but need not be symmetrical, e.g., 85% to 105%.

In another embodiment, the toxicity of the oxycodone prodrug is substantially lower than that of the unbound oxycodone. For example, in a preferred embodiment, the acute toxicity is 1-fold, 2-fold, 3-fold, 4-fold, 5-fold, 6-fold, 7-fold, 8-fold, 9-fold, 10-fold less, or increments therein less lethal than oral administration of unbound oxycodone.

For each of the described embodiments one or more characteristics as described throughout the specification may be realized. It should also be recognized that the compounds and compositions described throughout the specification may be utilized for a variety of novel methods of treatment, reduction of abuse potential, reduction of toxicity, improved release profiles, etc. An embodiment may obtain, one or more of a conjugate with toxicity of oxycodone that is substantially lower than that of unbound oxycodone; a conjugate where the covalently bound chemical moiety reduces or eliminates the possibility of overdose by oral administration; a conjugate where the covalently bound chemical moiety reduces or eliminates the possibility of overdose by intranasal administration; and/or a conjugate where the covalently bound chemical moiety reduces or eliminates the possibility of overdose by injection.

In accordance with the invention and as used herein, the following terms are defined with the following meanings, unless explicitly stated otherwise.

The compounds, compositions and methods of the invention utilize “oxycodone conjugates,” which are also referred to as oxycodone prodrugs.

Throughout this application the use of “chemical moiety”—sometimes referred to as the “conjugate” or the “carrier”—is meant to include any chemical substance, naturally occurring or synthetic that decreases the pharmacological activity until the oxycodone is released including at least carrier peptides, glycopeptides, carbohydrates, lipids, nucleic acids, nucleosides, or vitamins. Preferably, the chemical moiety is generally recognized as safe (“GRAS”).

Throughout this application the use of “carrier peptide” is meant to include naturally occurring amino acids, synthetic amino acids, and combinations thereof. In particular, carrier peptide is meant to include at least a single amino acid, a dipeptide, a tripeptide, an oligopeptide, a polypeptide, or the nucleic acid-amino acids peptides. The carrier peptide can comprise a homopolymer or heteropolymer of naturally occurring or synthetic amino acids.

The use of the term “straight carrier peptide” is meant to include amino acids that are linked via a —C(O)—NH— linkage, also referred to herein as a “peptide bond,” but may be substituted along the side chains of the carrier peptide. Amino acids that are not joined together via a peptide bond or are not exclusively joined through peptide bonds are not meant to fall within the definition of straight carrier peptide.

The use of the term “unsubstituted carrier peptide” is meant to include amino acids that are linked via a —C(O)—NH— linkage, and are not otherwise substituted along the side chains of the carrier peptide. Amino acids that are not joined together via a peptide bond or are not exclusively joined through peptide bonds are not meant to fall within the definition of unsubstituted carrier peptide.

“Oligopeptide” is meant to include from 2 amino acids to 10 amino acids. “Polypeptides” are meant to include from 2 to 50 amino acids.

“Carbohydrates” includes sugars, starches, cellulose, and related compounds. More specific examples include for instance, fructose, glucose, lactose, maltose, sucrose, glyceraldehyde, dihydroxyacetone, erythrose, ribose, ribulose, xylulose, galactose, mannose, sedoheptulose, neuraminic acid, dextrin, and glycogen.

A “glycoprotein” is a compound containing carbohydrate (or glycan) covalently linked to protein. The carbohydrate may be in the form of a monosaccharide, disaccharide(s), oligosaccharide(s), polysaccharide(s), or their derivatives (e.g. sulfo- or phospho-substituted).

A “glycopeptide” is a compound consisting of carbohydrate linked to an oligopeptide composed of L- and/or D-amino acids. A glyco-amino-acid is a saccharide attached to a single amino acid by any kind of covalent bond. A glycosyl-amino-acid is a compound consisting of saccharide linked through a glycosyl linkage (O—, N— or S—) to an amino acid.

The “carrier range” or “carrier size” is determined based on the effect desired. It is preferably between one to 12 chemical moieties with one to 8 moieties being preferred. In another embodiment the number of chemical moieties attached is a specific number e.g., 1, 2, 3, 4, 5, 6, 7, 8, 9, or 10, etc. Alternatively, the chemical moiety may be described based on its molecular weight. It is preferred that the conjugate weight is below about 2,500 kD, more preferably below about 1,500 kD.

A “composition” as used herein, refers broadly to any composition containing a oxycodone conjugate. A “pharmaceutical composition” refers to any composition containing a oxycodone conjugate that only comprises components that are acceptable for pharmaceutical uses, e.g., excludes oxycodone conjugates for immunological purposes.

Use of phrases such as “decreased”, “reduced”, “diminished”, or “lowered” includes at least a 10% change in pharmacological activity with respect to at least one ADME characteristic or at least one of AUC, Cₘₐₓ, Tₘₐₓ, Cₘᵢₙ, and t_{1/2} with greater percentage changes being preferred for reduction in abuse potential and overdose potential. For instance, the change may also be greater than 25%, 35%, 45%, 55%, 65%, 75%, 85%, 95%, 96%, 97%, 98%, 99%, or other increments.

Use of the phrase “similar pharmacological activity” means that two compounds exhibit curves that have substantially the same AUC, Cₘₐₓ, Tₘₐₓ, Cₘᵢₙ, and/or t_{1/2} parameters, preferably within about 30% of each other, more preferably within about 25%, 20%, 10%, 5%, 2%, 1%, or other increments.

“Cₘₐₓ” is defined as the maximum concentration of free oxycodone in the body obtained during the dosing interval.

“Tₘₐₓ” is defined as the time to maximum concentration.

“Cₘᵢₙ” is defined as the minimum concentration of oxycodone in the body after dosing.

“t_{1/2}” is defined as the time required for the amount of oxycodone in the body to be reduced to one half of its value.

Throughout this application, the term “increment” is used to define a numerical value in varying degrees of precision, e.g., to the nearest 10, 1, 0.1, 0.01, etc. The increment can be rounded to any measurable degree of precision. For example, the range 1 to 100 or increments therein includes ranges such as 20 to 80, 5 to 50, 0.4 to 98, and 0.04 to 98.05.

“Acute pain” is defined as sharp or severe pain or discomfort that lasts for a short period of time. Preferably, a short period of time is less than 3 months for nociceptive or neurogenic pain, and less than 6 months for psychogenic pain.

“Chronic pain” is defined as moderate to severe pain that lasts for a long period of time. Preferably, a long period of time is more than 3 months for nociceptive or neurogenic pain and more than 6 months for psychogenic pain.

Patient” as used herein, refers broadly to any animal that is in need of treatment, most preferably and animal that is in pain. The patient may be a clinical patient such as a human or a veterinary patient such as a companion, domesticated, livestock, exotic, or zoo animal. Animals may be mammals, reptiles, birds, amphibians, or invertebrates.

“Mammal” as used herein, refers broadly to any and all warm-blooded vertebrate animals of the class Mammalia, including humans, non-human primates, felines, canines, pigs, horses, sheep, etc.

“Pretreatment” as used herein, refers broadly to any and all preparation, treatment, or protocol that takes place before receiving a oxycodone compound or composition of the invention.

“Treating” or “treatment” as used herein, refers broadly to preventing the disease, i.e., causing the clinical symptoms of the disease not to develop in a patient that may be exposed to or predisposed to the disease but does not yet experience or display symptoms of the disease, inhibiting the disease, i.e., arresting or reducing the development of the disease or its clinical symptoms, and/or relieving the disease, i.e., causing regression of the disease or its clinical symptoms. Treatment also encompasses an alleviation of signs and/or symptoms.

“Therapeutically effective amount” as used herein, refers broadly to the amount of a compound that, when administered to a patient for treating pain is sufficient to effect such treatment for pain. The “therapeutically effective amount” will vary depending on the compound, the disease and its severity and the age, weight, etc., of the patient to be treated. “Effective dosage” or “Effective amount” of the oxycodone compound or composition is that which is necessary to treat or provide prophylaxis for oxycodone.

“Selection of patients” and “Screening of patients” as used herein, refers broadly to the practice of selecting appropriate patients to receive the treatments described herein. Various factors including but not limited to age, weight, heath history, medications, surgeries, injuries, conditions, illnesses, diseases, infections, gender, ethnicity, genetic markers, polymorphisms, skin color, and sensitivity to hydromorphone treatment. Still other factors include those used by physicians to determine if a patient is appropriate to receive the treatments described herein.

“Diagnosis” as used herein, refers broadly to the practice of testing, assessing, assaying, and determining whether or not a patient is in pain.

Regarding stereochemistry, this patent is meant to cover all compounds discussed regardless of absolute configurations. Thus, natural, L-amino acids are discussed but the use of D-amino acids are also included, but not preferred.

For each of the embodiments recited herein, the carrier peptide may comprise of one or more of the naturally occurring (L-) amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine. Other preferred amino acids include beta-alanine, beta-leucine and tertiary-leucine. In another embodiment the amino acid or peptide is comprised of one or more of the D-form of the naturally occurring amino acids. In another embodiment the amino acid or peptide is comprised of one or more unnatural, non-standard or synthetic amino acids such as, aminohexanoic acid, biphenylalanine, cyclohexylalanine, cyclohexylglycine, diethylglycine, dipropylglycine, 2,3-diaminoproprionic acid, homophenylalanine, homoserine, homotyrosine, naphthylalanine, norleucine, ornithine, pheylalanine(4-fluoro), phenylalanine(2,3,4,5,6 pentafluoro), phenylalanine(4-nitro), phenylglycine, pipecolic acid, sarcosine, tetrahydroisoquinoline-3-carboxylic acid, and tert-leucine. In another embodiment the amino acid or peptide comprises of one or more amino acid alcohols. In another embodiment the amino acid or peptide comprises of one or more N-methyl amino acids.

In another embodiment, the specific carriers listed in the table may have one or more of amino acids substituted with one of the 20 naturally occurring amino acids. It is preferred that the substitution be with an amino acid which is similar in structure or charge compared to the amino acid in the sequence. For instance, isoleucine (Ile)[I] is structurally very similar to leucine (Leu)[L], whereas, tyrosine (Tyr)[Y] is similar to phenylalanine (Phe)[F], whereas serine (Ser)[S] is similar to threonine (Thr)[T], whereas cysteine (Cys)[C] is similar to methionine (Met)[M], whereas alanine (Ala)[A] is similar to valine (Val)[V], whereas lysine (Lys)[K] is similar to arginine (Arg)[R], whereas asparagine (Asn)[N] is similar to glutamine (Gln)[Q], whereas aspartic acid (Asp)[D] is similar to glutamic acid (Glu)[E], whereas histidine (His)[H] is similar to proline (Pro)[P], and glycine (Gly)[G] is similar to tryptophan (Trp)[W]. In the alternative the preferred amino acid substitutions may be selected according to hydrophilic properties (i.e., polarity) or other common characteristics associated with the 20 essential amino acids. While preferred embodiments utilize the 20 natural amino acids for their GRAS characteristics, it is recognized that minor substitutions along the amino acid chain that do not affect the essential characteristics of the amino are also contemplated.

The oxycodone conjugate may also be in salt form. Pharmaceutically acceptable salts, e.g., non-toxic, inorganic and organic acid addition salts, are known in the art. Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, bisulfate, bitartrate, borate, butyrate, calcium edetate, camphorate, camphorsulfonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, isothionate, lactate, lactobionate, laurate, laurylsulphonate, malate, maleate, mandelate, methanesulfonate, mucate, naphthylate, napsylate, nicotinate, N-methylglucamine ammonium salt, oleate, palmitate, pamoate, pantothenate, pectinate, phosphate, phosphateldiphosphate, pivalate, polygalacturonate, propionate, p-toluenesulfonate, saccharate, salicylate, stearate, subacetate, succinate, sulfate, sulfosaliculate, suramate, tannate, tartrate, teoclate, tosylate, triethiodide, undecanoate, and valerate salts, and the like.

In the invention, oxycodone may be covalently attached to the peptide via a ketone group and a linker. This linker may be a small linear or cyclic molecule containing 2-6 atoms with one or more heteroatoms (such as O, S, N) and one or more functional groups (such as amines, amides, alcohols or acids) or may be made up of a short chain of either amino acids or carbohydrates). For example, glucose would be suitable as a linker.

In yet another embodiment of the invention, linkers can be selected from the group of all chemical classes of compounds such that virtually any side chain of the peptide can be attached. The linker should have a functional pendant group, such as a carboxylate, an alcohol, thiol, oxime, hydrazone, hydrazide, or an amine group, to covalently attach to the carrier peptide. In one preferred embodiment, the alcohol group of oxycodone is covalently attached to the N-terminus of the peptide via a linker. In another preferred embodiment the ketone group of oxycodone is attached to a linker through the formation of a ketal and the linker has a pendant group that is attached to the carrier peptide.

Additionally information regarding the attachment of active agents such as oxycodone to carriers may be found in U.S. Pat. No. 7,060,708, and/or PCT/US03/05524 (WO 03/079972 A1), and/or PCT/US03/05525 (WO 03/072046 A1), and/or U.S. Patent Application Publication US 2005/0176644 A1 each of which is hereby incorporated by reference in its entirety.

In addition to the oxycodone prodrug, the pharmaceutical compositions of the invention may further comprise one or more pharmaceutical additives. Pharmaceutical additives include a wide range of materials including, but not limited to diluents and bulking substances, binders and adhesives, lubricants, glidants, plasticizers, disintegrants, carrier solvents, buffers, colorants, flavorings, sweeteners, preservatives and stabilizers, adsorbents, and other pharmaceutical additives known in the art.

Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil.

Surface agents for formulation include, but are not limited to, sodium lauryl sulfate, dioctyl sodium sulfosuccinate, triethanolamine, polyoxyethylene sorbitan, poloxalkol, and quarternary ammonium salts; excipients such as lactose, mannitol, glucose, fructose, xylose, galactose, sucrose, maltose, xylitol, sorbitol, chloride, sulfate and phosphate salts of potassium, sodium, and magnesium; gelling agents such as colloidal clays; thickening agents such as gum tragacanth or sodium alginate, effervescing mixtures; and wetting agents such as lecithin, polysorbates or laurylsulphates.

Colorants can be used to improve appearance or to help identify the pharmaceutical composition. See 21 C.F.R., Part 74. Exemplary colorants include D&C Red No. 28, D&C Yellow No. 10, FD&C Blue No. 1, FD&C Red No. 40, FD&C Green #3, FD&C Yellow No. 6, and edible inks.

In embodiments where the pharmaceutical composition is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate, as well as other conventional binders known to persons skilled in the art. Exemplary non-limiting bulking substances include sugar, lactose, gelatin, starch, and silicon dioxide.

Glidants can improve the flowability of non-compacted solid dosage forms and can improve the accuracy of dosing. Glidants include, but are not limited to, colloidal silicon dioxide, fumed silicon dioxide, silica gel, talc, magnesium trisilicate, magnesium or calcium stearate, powdered cellulose, starch, and tribasic calcium phosphate.

Plasticizers include, but are not limited to, hydrophobic and/or hydrophilic plasticizers such as, diethyl phthalate, butyl phthalate, diethyl sebacate, dibutyl sebacate, triethyl citrate, acetyltriethyl citrate, acetyltributyl citrate, cronotic acid, propylene glycol, castor oil, triacetin, polyethylene glycol, propylene glycol, glycerin, and sorbitol. Plasticizers are particularly useful for pharmaceutical compositions containing a polymer and in soft capsules and film-coated tablets.

Flavorings improve palatability and may be particularly useful for chewable tablet or liquid dosage forms. Flavorings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

Preservatives and/or stabilizers improving storagability include, but are not limited to, alcohol, sodium benzoate, butylated hydroxy toluene, butylated hydroxyanisole, and ethylenediamine tetraacetic acid.

Disintegrants can increase the dissolution rate of a pharmaceutical composition. Disintegrants include, but are not limited to, alginates such as alginic acid and sodium alginate, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), polyvinylpolypyrrolidine (Plasone-XL®), guar gum, magnesium aluminum silicate, methyl cellulose, microcrystalline cellulose, polacrilin potassium, powdered cellulose, starch, pregelatinized starch, sodium starch glycolate (e.g., Explotab®, Primogel®).

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc.

In embodiments where the pharmaceutical composition is formulated for a liquid dosage form, the pharmaceutical composition may include one or more solvents. Suitable solvents include, but are not limited to, water; alcohols such as ethanol and isopropyl alcohol; vegetable oil; polyethylene glycol; propylene glycol; and glycerin or mixing and combination thereof.

The pharmaceutical composition can comprise a buffer. Buffers include, but are not limited to, lactic acid, citric acid, acetic acid, sodium lactate, sodium citrate, and sodium acetate.

Hydrophilic polymers suitable for use in the sustained release formulation include: one or more natural or partially or totally synthetic hydrophilic gums such as acacia, gum tragacanth, locust bean gum, guar gum, or karaya gum, modified cellulosic substances such as methylcellulose, hydroxomethylcellulose, hydroxypropyl methylcellulose, hydroxypropyl cellulose, hydroxyethylcellulose, carboxymethylcellulose; proteinaceous substances such as agar, pectin, carrageen, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art or a combination of such polymers.

One of ordinary skill in the art would recognize a variety of structures, such as bead constructions and coatings, useful for achieving particular release profiles. It is also possible for the dosage form to combine any forms of release known to persons of ordinary skill in the art. These include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is known in the art. See, e.g., U.S. Pat. No. 6,913,768.

However, it should be noted that the oxycodone conjugate controls the release of oxycodone into the digestive tract over an extended period of time resulting in an improved profile when compared to immediate release combinations and reduces and/or prevents abuse without the addition of the above additives. In a preferred embodiment no further sustained release additives are required to achieve a blunted or reduced pharmacokinetic curve (e.g. reduced euphoric effect) while achieving therapeutically effective amounts of oxycodone release.

The dose range for adult human beings will depend on a number of factors including the age, weight and condition of the patient and the administration route. Tablets and other forms of presentation provided in discrete units conveniently contain a daily dose, or an appropriate fraction thereof, of the oxycodone conjugate. The dosage form can contain a dose of about 2.5 mg to about 500 mg, about 10 mg to about 250 mg, about 10 mg to about 100 mg, about 25 mg to about 75 mg, or increments therein. In a preferred embodiment, the dosage form contains 30 mg, 50 mg, or 70 mg of a oxycodone prodrug.

Tablets and other dosage forms provided in discrete units can contain a daily dose, or an appropriate fraction thereof, of one or more oxycodone prodrugs.

Compositions of the invention may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24-hour period. The doses may be uneven doses with regard to one another or with regard to the individual components at different administration times. Preferably, a single dose is administered once daily.

Likewise, the compositions of the invention may be provided in a blister pack or other such pharmaceutical package. Further, the compositions of the present inventive subject matter may further include or be accompanied by indicia allowing individuals to identify the compositions as products for a prescribed treatment. The indicia may further additionally include an indication of the above specified time periods for administering the compositions. For example the indicia may be time indicia indicating a specific or general time of day for administration of the composition, or the indicia may be a day indicia indicating a day of the week for administration of the composition. The blister pack or other combination package may also include a second pharmaceutical product.

The compounds of the invention can be administered by a variety of dosage forms. Any biologically acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspension in an aqueous liquid or a non-aqueous liquid, emulsions, tablets, syringes, multi-layer tablets, bi-layer tablets, capsules, soft gelatin capsules, hard gelatin capsules, caplets, lozenges, chewable lozenges, beads, powders, granules, particles, microparticles, dispersible granules, cachets, infusions, emulsions, health bars, confections, animal feeds, cereals, yogurts, cereal coatings, foods, nutritive foods, functional foods and combinations thereof. Preferably, said composition may be in the form of any of the known varieties of tablets (e.g., chewable tablets, conventional tablets, film-coated tablets, compressed tablets), capsules, liquid dispersions for oral administration (e.g., syrups, emulsions, solutions or suspensions).

However, the most effective means for delivering the abuse-resistant oxycodone compounds of the invention is orally, to permit maximum release of oxycodone to provide therapeutic effectiveness and/or sustained release while maintaining abuse resistance. When delivered by the oral route oxycodone is released into circulation, preferably over an extended period of time as compared to oxycodone alone.

It is preferred that the oxycodone conjugate be compact enough to allow for a reduction in overall administration size. The smaller size of the oxycodone prodrug dosage forms promotes ease of swallowing.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable or necessary, flavoring, preserving, suspending, thickening or emulsifying agents can be included.

Accordingly, the invention also provides methods comprising providing, administering, prescribing, or consuming a oxycodone prodrug. The invention also provides pharmaceutical compositions comprising a oxycodone prodrug. The formulation of such a pharmaceutical composition can optionally enhance or achieve the desired release profile.

### EXAMPLES

Any feature of the above-describe embodiments can be used in combination with any other feature of the above-described embodiments.

In order to facilitate a more complete understanding of the invention, Examples are provided below. However, the scope of the invention is not limited to specific embodiments disclosed in these Examples, which are for purposes of illustration only.

Table 1 lists exemplary carrier peptides to which oxycodone may be covalently bonded.

TABLE 1
List of Preferred Amino Acids and Peptides to which
Oxycodone may be covalently bonded.

| | | | |
|---|---|---|---|
| Ala | Glu-Val-Val | Phe-Ser-Val | Tyr-Tyr-Phe |
| Arg | Gly-Asp-Val | Phe-Thr-Val | Tyr-Tyr-Val |
| Asn | Gly-Gly-Cha | Phe-Tyr-Val | Tyr-Val-Val |
| Asp | Gly-Gly-hPhe | Pro-Asp-Val | Val-Asp-Val |
| Cys | Gly-Gly-Ile | Pro-Gly-Val | Val-Gln-Val |
| Gln | Gly-Gly-Leu | Pro-Ile-Ile | Val-Glu-Gly |
| Glu | Gly-Pro-Val | Pro-Ile-Val | Val-Glu-Leu |
| Gly | Gly-Ser-Val | Pro-Leu-Ile | Val-Glu-Val |
| His | Gly-Thr-Val | Pro-Lys-Val | Val-Gly-Glu |
| Ile | Gly-Val-Val | Pro-Phe-Ile | Val-Gly-Val |
| Leu | Gly-Gly-Nle | Pro-Phe-Val | Val-Phe-Val |
| Lys | Gly-Gly-Phe | Pro-Pro-Cha | Val-Pro-Tyr |
| Met | Gly-Gly-Val | Pro-Pro-Ile | Val-Pro-Val |
| Phe | Gly-Ile-Ile | Pro-Pro-Leu | Val-Thr-Val |
| Pro | Gly-Lys-Val | Pro-Pro-Nle | Val-Tyr-Asp |
| Ser | Ile-Asp-Val | Pro-Pro-Phe | Val-Tyr-Asp |
| β-Leu | Ile-Glu-Val | Pro-Pro-Val | Val-Tyr-Glu |
| Thr | Ile-Gly-Val | Pro-Pro-Val | Val-Tyr-Gly |
| t-Leu | Ile-Phe-Val | Pro-Ser-Val | Val-Tyr-Ile |
| Trp | Ile-Ser-Val | Pro-Thr-Val | Val-Tyr-Leu |
| Tyr | Ile-Thr-Val | Pro-Tyr-Val | Val-Tyr-Lys |
| Val | Ile-Tyr-Val | Pro-Tyr-Val | Val-Tyr-Phe |
| β-Ala | Leu-Asp-Val | Pro-Val-Val | Val-Tyr-Pro |
| Glu_{pyro}-Glu | Leu-Glu-Val | Ser-Asp-Val | Val-Tyr-Val |
| Tyr-β-Ala | Leu-Gly-Val | Ser-Glu-Val | Lys-Tyr-Val-Ile |
| | | | [SEQ ID NO: 1] |
| β-Ala-β-Ala | Leu-Leu-Ile | Ser-Gly-Val | Tyr-Pro-Val-Ile |
| | | | [SEQ ID NO: 2] |
| Asp-Asp-Cha | Leu-Lys-Val | Ser-Ile-Val | Acetyl-Glu₂-Pro₂- |
| | | | Ile |
| | | | [SEQ ID NO: 3] |
| Asp-Asp-Ile | Leu-Phe-Val | Ser-Leu-Val | Asp₂-Gly₂-Ile |
| | | | [SEQ ID NO: 4] |
| Asp-Asp-Nle | Leu-Pro-Ile | Ser-Lys-Val | Asp₂-Leu₂-Ile |
| | | | [SEQ ID NO: 5] |
| Asp-Asp-Phe | Leu-Pro-Val | Ser-Phe-Val | Asp₂-Leu₂-Ile |
| | | | [SEQ ID NO: 6] |
| Asp-Asp-Val | Leu-Thr-Val | Ser-Pro-Val | Asp₂-Pro₂-Ile |
| | | | [SEQ ID NO: 7] |
| Asp-d-Asp-Ile | Leu-Tyr-Val | S er-Tyr-Val | Glu₂-Gly₂-Phe |
| | | | [SEQ ID NO: 8] |
| Asp-Glu-Val | Lys-Asp-Val | Ser-Val-Val | Glu₂-Leu₃ |
| | | | [SEQ ID NO: 9] |
| Asp-Gly-Val | Lys-Glu-Val | Thr-Asp-Val | Glu₂-Phe₂-Leu |
| | | | [SEQ ID NO: 10] |
| Asp-Ile-Val | Lys-Gly-Val | Thr-Glu-Val | Glu₂-Phe-Pro-Ile |
| | | | [SEQ ID NO: 11] |
| Asp-Leu-Val | Lys-Ile-Val | Thr-Gly-Val | Glu₂-Pro₂-Leu |
| | | | [SEQ ID NO: 12] |
| Asp-Lys-Val | Lys-Leu-Val | Thr-Leu-Val | Glu₂-Pro-Phe-Ile |
| | | | [SEQ ID NO: 13] |
| Asp-Phe-Val | Lys-Lys-Ile | Thr-Lys-Val | Glu₄-Ile |
| | | | [SEQ ID NO: 14] |
| Asp-Pro-Val | Lys-Lys-Leu | Thr-Phe-Val | Glu-Glu-Phe-Phe- |
| | | | Phe |
| | | | [SEQ ID NO: 15] |
| Asp-Ser-Val | Lys-Lys-Val | Thr-Pro-Val | Gly₂-Glu₂-Ile |
| | | | [SEQ ID NO: 16] |
| Asp-Thr-Val | Lys-Phe-Val | Thr-Ser-Val | Lys₂-Leu₂-Ile |
| | | | [SEQ ID NO: 17] |
| Asp-Tyr-Val | Lys-Pro-Val | Thr-Thr-Ile | Lys₂-Pro₂-Ile |
| | | | [SEQ ID NO: 18] |
| Asp-Val-Val | Lys-Thr-Val | Thr-Thr-Val | Phe₂-Glu₂-Ile |
| | | | [SEQ ID NO: 19] |
| Gln-Gln-Ile | Lys-Tyr-Val | Thr-Tyr-Val | Phe₅ |
| | | | [SEQ ID NO: 20] |
| Gln-Gln-Val | Lys-Tyr-Val | Thr-Val-Val | Thr₂-Gly₂-Ile |
| | | | [SEQ ID NO: 21] |
| Gln-Gln-β-Ala | Lys-Val-Val | Tyr-Asp-Val | Thr₂-Phe₂-Ile |
| | | | [SEQ ID NO: 22] |
| Gln-Pro-Val | Phe-Asp-Val | Tyr-Glu-Val | Tyr₂-Leu₂-Ile |
| | | | [SEQ ID NO: 23] |
| Glu-Glu-Cha | Phe-Glu-Val | Tyr-Gly-Val | Tyr₂-Phe₂-Ile |
| | | | [SEQ ID NO: 24] |
| Glu-Glu-hPhe | Phe-Gly-Val | Tyr-Ile-Val | Tyr₂-Pro₂-Ile |
| | | | [SEQ ID NO: 25] |
| Glu-Glu-Ile | Phe-Ile-Val | Tyr-Leu-Val | Tyr₂-Pro-Phe-Ile |
| | | | [SEQ ID NO: 26] |
| Glu-Glu-Leu | Phe-Leu-Val | Tyr-Lys-Val | Tyr-Tyr-Phe-Phe- |
| | | | Ile |
| | | | [SEQ ID NO: 27] |
| Glu-Glu-Nle | Phe-Lys-Val | Tyr-Phe-Val | Tyr-Tyr-Phe-Phe- |
| | | | Val |
| | | | [SEQ ID NO: 28] |
| Glu-Glu-Phe | Phe-Phe-Cha | Tyr-Pro-Val | Asp₂-Lys(Asp₂) |
| | | | [SEQ ID NO: 29] |
| Glu-Glu-Val | Phe-Phe-hPhe | Tyr-Ser-Val | Glu₂-Lys(Glu₂) |
| | | | [SEQ ID NO: 30] |
| Glu-Gly-Val | Phe-Phe-Ile | Tyr-Thr-Val | Phe₂-Lys(Phe₂) |
| | | | [SEQ ID NO: 31] |
| Glu-Leu-Val | Phe-Phe-Leu | Tyr-Tyr-Ala | Pro₂-Lys(Pro₂) |
| | | | [SEQ ID NO: 32] |
| Glu-Lys-Val | Phe-Phe-Nle | Tyr-Tyr-Cha | Tyr₂-Lys(Tyr₂) |
| | | | [SEQ ID NO: 33] |
| Glu-Phe-Val | Phe-Phe-Phe | Tyr-Tyr-hPhe | Ethyl Carbonate |
| Glu-Ser-Val | Phe-Phe-Val | Tyr-Tyr-Ile | galactose-Gly-Gly- |
| | | | Ile |
| Glu-Thr-Val | Phe-Phe-Val | Tyr-Tyr-Leu | galactose-Gly-Gly- |
| | | | Leu |
| Glu-Tyr-Val | Phe-Pro-Val | Tyr-Tyr-Nle | galactose-Ile |

Referring to Table 1, it is noted that for disubstituted conjugates, each of the sequences listed above may be present along with any other sequence to form a disubstituted oxycodone conjugate. In addition, a disubstituted oxycodone conjugate may be formed from substitution at two positions with two occurrences of one of the above sequences.

The following Table lists preferred oxycodone conjugates made according to the invention. The designation [peptide]₂-OC refers to a disubstituted oxycodone conjugate according to Structure (V) set forth above. In addition, the designation [peptide]-OC-[peptide] refers to a disubstituted oxycodone conjugate, wherein the peptide that precedes OC is bound to the 6 position of oxycodone and the peptide the follows OC is at the 14 position.

TABLE 2
List of Oxycodone (OC) Conjugates attached through the 6 position (and
also through the 14 position for disubstituted OC conjugates) to the C-terminus
of the amino acid according to the invention (for clarity purposes the amino
acid that is next to the -OC is the amino acid that is connected to the OC).

| | | |
|---|---|---|
| β-alanine-OC | [Asp-Lys-Val]₂-OC | [Pro-Asp-Val]₂-OC |
| Glu-OC | [Asp-Phe-Val]₂-OC | [Pro-Val-Val]₂-OC |
| Ile-OC | [Asp-Pro-Val]₂-OC | [Ser-Thr-Val]₂-OC |
| Leu-OC | [Asp-Ser-Val]₂-OC | [Ser-Asp-Val]₂-OC |
| Phe-OC | [Asp-Thr-Val]₂-OC | [Ser-Glu-Val]₂-OC |
| β-Leu-OC | [Asp-Tyr-Val]₂-OC | [Ser-Gly-Val]₂-OC |
| Val-OC | [Asp-Val-Val]₂-OC | [Ser-Ile-Val]₂-OC |
| β-Ala-β-Ala-OC | [Bio-Gly₂-Ile]₂-OC | [Ser-Leu-Val]₂-OC |
| Tyr-β-Ala-OC | [Bio-Gly₂-Leu]₂-OC | [Ser-Lys-Val]₂-OC |
| Asp-Asp-Ile-OC | [Gal-Gly₂-Ile]₂-OC | [Ser-Phe-Val]₂-OC |
| Asp-Asp-Val-OC | [Gal-Gly₂-Leu]₂-OC | [Ser-Pro-Val]₂-OC |
| Ala-Ala-Val-OC | [Gal-Pro₂-Ile]₂-OC | [Ser-Tyr-Val]₂-OC |
| Gln-Gln-β-Ala-OC | [Gal-Pro₂-Leu]₂-OC | [Ser-Val-Val]₂-OC |
| Gln-Gln-Ile-OC | [Gln-Gln-Val]₂-OC | |
| Glu-Leu-Val-OC | [Gln-Pro-Val]₂-OC | |
| Glu-Tyr-Val-OC | [Glu-Asp-Val]₂-OC | [Thr-Thr-Val]₂-OC |
| Glu-Glu-Ala-OC | [Glu-Glu-Cha]₂-OC | [Thr-Asp-Val]₂-OC |
| Glu-Glu-Ile-OC | [Glu-Glu-hPhe]₂-OC | [Thr-Glu-Val]₂-OC |
| Glu-Glu-Leu-OC | [Glu-Glu-Nle]₂-OC | [Thr-Gly-Val]₂-OC |
| Glu-Glu-Phe-OC | [Glu-Glu-Phe]₂-OC | [Thr-Leu-Val]₂-OC |
| Glu-Glu-Pro-OC | [Glu-Gly-Val]₂-OC | [Thr-Lys-Val]₂-OC |
| Glu-Glu-β-Ala-OC | [Glu-Leu-Val]₂-OC | [Thr-Phe-Val]₂-OC |
| Glu-Glu-Val-OC | [Glu-Lys-Val]₂-OC | [Thr-Pro-Val]₂-OC |
| Glu-Tyr-Val-OC-OAc | [Glu-Phe-Val]₂-OC | [Thr-Ser-Val]₂-OC |
| Glu-Tyr-Val-OC-OCOOEt | [Glu-Pro-Val]₂-OC | [Thr-Tyr-Val]₂-OC |
| Gly-Gly-Ile-OC | [Glu-Ser-Val]₂-OC | [Thr-Val-Val]₂-OC |
| Gly-Gly-Leu-OC | [Glu-Thr-Val]₂-OC | [Tyr-Pro-Val]₂-OC |
| Gly-Gly-Phe-OC | [Glu-Tyr-Val]₂-OC | [Tyr-Tyr-Cha]₂-OC |
| Gly-Gly-β-Ala-OC | [Glu-Val-Val]₂-OC | [Tyr-Tyr-hPhe]₂-OC |
| Gly-Gly-Val-OC | [Gly-Gly-Cha]₂-OC | [Tyr-Tyr-Nle]₂-OC |
| Ile-Ile-Ile-OC | [Gly-Gly-hPhe]₂-OC | [Tyr-Tyr-Phe]₂-OC |
| Ile-Tyr-Val-OC | [Gly-Gly-Nle]₂-OC | [Tyr-Asp-Val]₂-OC |
| Ile-Tyr-Val-OC-OAc | [Gly-Gly-Phe]₂-OC | [Tyr-Glu-Val]₂-OC |
| Ile-Tyr-Val-OC-OCOOEt | [Gly-Gly-Val]₂-OC | [Tyr-Gly-Val]₂-OC |
| Leu-Leu-Ala-OC | [Gly-Leu-Val]₂-OC | [Tyr-Ile-Val]₂-OC |
| Leu-Leu-Ile-OC | [Gly-Asp-Val]₂-OC | [Tyr-Leu-Val]₂-OC |
| Leu-Leu-Leu-OC | [Gly-Glu-Val]₂-OC | [Tyr-Lys-Val]₂-OC |
| Leu-Leu-Val-OC | [Gly-Lys-Val]₂-OC | [Tyr-Phe-Val]₂-OC |
| Leu-Leu-β-Ala-OC | [Gly-Phe-Val]₂-OC | [Tyr-Ser-Val]₂-OC |
| Leu-Tyr-Val-OC | [Gly-Pro-Val]₂-OC | [Tyr-Thr-Val]₂-OC |
| Lys-Lys-Ala-OC | [Gly-Ser-Val]₂-OC | [Tyr-Tyr-Val]₂-OC |
| Lys-Lys-Ile-OC | [Gly-Thr-Val]₂-OC | [Tyr-Val-Val]₂-OC |
| Lys-Lys-Leu-OC | [Gly-Tyr-Val]₂-OC | [Val-Glu-Val]₂-OC |
| | | [Val-Gln-Val]₂-OC |
| Lys-Lys-Phe-OC | [Gly-Val-Val]₂-OC | [Val-Asp-Val]₂-OC |
| Lys-Lys-Val-OC | [Ile-Tyr-Val]₂-OC | [Val-Glu-Val]₂-OC |
| Lys-Lys-β-Ala-OC | [Ile-Asp-Val]₂-OC | [Val-Gly-Val]₂-OC |
| Lys-Tyr-Val-OC-OAc | [Ile-Glu-Val]₂-OC | [Val-Phe-Val]₂-OC |
| Lys-Tyr-Val-OC-OCOOEt | [Ile-Gly-Val]₂-OC | [Val-Pro-Val]₂-OC |
| Phe-Phe-Leu-OC | [Ile-Phe-Val]₂-OC | [Val-Thr-Val]₂-OC |
| Phe-Phe-Ile-OC | [Ile-Ser-Val]₂-OC | [Val-Tyr-Val]₂-OC |
| Phe-Phe-Val-OC | [Ile-Thr-Val]₂-OC | Ile-Tyr-Val-OC-Val-Glu-Val |
| Phe-Tyr-Val-OC | [Leu-Gly-Val]₂-OC | Ile-Tyr-Val-OC-Val-Gly-Glu |
| Pro2-Ile-OC | [Leu-Lys-Val]₂-OC | Ile-Tyr-Val-OC-Val-Pro-Tyr |
| Pro2-Leu-OC | [Leu-Phe-Val]₂-OC | Ile-Tyr-Val-OC-Val-Tyr-Asp |
| Pro-Glu-Val-OC | [Leu-Pro-Val]₂-OC | Ile-Tyr-Val-OC-Val-Tyr-Glu |
| Pro-Pro-Ala-OC | [Leu-Thr-Val]₂-OC | Ile-Tyr-Val-OC-Val-Tyr-Gly |
| Pro-Pro-Ile-OC | [Leu-Tyr-Val]₂-OC | Ile-Tyr-Val-OC-Val-Tyr-Lys |
| Pro-Pro-Leu-OC | [Lys-Lys-Val]₂-OC | Ile-Tyr-Val-OC-Val-Tyr-Pro |
| Pro-Pro-Val-OC | [Lys-Ser-Val]₂-OC | Leu-Tyr-Val-OC-Gly-Tyr-Leu |
| Pro-Tyr-Val-OC | [Lys-Asp-Val]₂-OC | Leu-Tyr-Val-OC-Val-Glu-Gly |
| Ser-Ser-Ser-OC | [Lys-Glu-Val]₂-OC | Ile-Tyr-Val-OC-Val-Glu-Leu |
| Thr-Thr-Thr-OC | [Lys-Gly-Val]₂-OC | Ile-Tyr-Val-OC-Gly-Tyr-Ile |
| Thr-Thr-Val-OC | [Lys-Ile-Val]₂-OC | Ile-Tyr-Val-OC-Val-Glu-Gly |
| Succcinate-OC | [Lys-Leu-Val]₂-OC | Leu-Tyr-Val-OC-Val-Glu-Leu |
| Tyr-Tyr-Ala-OC | [Lys-Phe-Val]₂-OC | Leu-Tyr-Val-OC-Val-Pro-Tyr |
| Tyr-Tyr-Ile-OC | [Lys-Pro-Val]₂-OC | Leu-Tyr-Val-OC-Val-Tyr-Gly |
| Tyr-Tyr-Leu-OC | [Lys-Thr-Val]₂-OC | Lys-Tyr-Val-OC-Val-Glu-Val |
| Tyr-Tyr-Phe-OC | [Lys-Tyr-Val]₂-OC | Lys-Tyr-Val-OC-Val-Gly-Glu |
| Tyr-Tyr-Pro-OC | [Lys-Val-Val]₂-OC | Lys-Tyr-Val-OC-Val-Tyr-Asp |
| Tyr-Tyr-β-Ala-OC | [Nia-Gly₂-Ile]₂-OC | Lys-Tyr-Val-OC-Val-Tyr-Glu |
| Tyr-Tyr-Val-OC | [Nia-Gly₂-Ile]₂-OC | Lys-Tyr-Val-OC-Val-Tyr-Ile |
| Val-Val-Leu-OC | [Nia-Gly₂-Leu]₂-OC | Lys-Tyr-Val-OC-Val-Tyr-Leu |
| Val-Val-Phe-OC | [Nia-Gly₂-Leu]₂-OC | Lys-Tyr-Val-OC-Val-Tyr-Lys |
| Val-Val-Val-OC | [Phe-Phe-Cha]₂-OC | Lys-Tyr-Val-OC-Val-Tyr-Phe |
| Lys-Tyr-Val-Ile-OC | [Phe-Phe-hPhe]₂-OC | Lys-Tyr-Val-OC-Val-Tyr-Pro |
| [SEQ ID NO: 1] | | |
| Tyr-Pro-Val-Ile-OC | [Phe-Phe-Nle]₂-OC | Lys-Tyr-Val-OC-Val-Tyr-Val |
| [SEQ ID NO: 2] | | |
| Glu-Glu-Phe-Phe-Ile-OC | [Phe-Phe-Phe]₂-OC | Phe-Tyr-Val-OC-Val-Glu-Gly |
| [SEQ ID NO: 34] | | |
| Glu-Glu-Phe-Phe-Phe-OC | [Phe-Phe-Val]₂-OC | Phe-Tyr-Val-OC-Val-Gly-Glu |
| [SEQ ID NO: 15] | | |
| Phe-Phe-Lys-Phe-Phe- OC | [Phe-Val-Val]₂-OC | Phe-Tyr-Val-OC-Val-Tyr-Asp |
| [SEQ ID NO: 31] | | |
| Tyr-Tyr-Lys-Tyr-Tyr-OC | [Phe-Asp-Val]₂-OC | Phe-Tyr-Val-OC-Val-Tyr-Glu |
| [SEQ ID NO: 33] | | |
| Tyr-Tyr-Phe-Phe-Ile-OC | [Phe-Glu-Val]₂-OC | Pro-Tyr-Val-OC-Val-Tyr-Glu |
| [SEQ ID NO: 27] | | |
| Tyr-Tyr-Phe-Phe-Val-OC | [Phe-Gly-Val]₂-OC | Pro-Tyr-Val-OC-Val-Tyr-lle |
| [SEQ ID NO: 28] | | |
| [Boc-Cha]₂-OC | [Phe-Ile-Val]₂-OC | Pro-Tyr-Val-OC-Val-Tyr-Leu |
| [Boc-Dpg]₂-OC | [Phe-Leu-Val]₂-OC | Tyr-Pro-Val-OC-Val-Tyr-Glu |
| [Boc-hPhe]₂-OC | [Phe-Lys-Val]₂-OC | Tyr-Pro-Val-OC-Val-Tyr-Ile |
| [Boc-Nle]₂-OC | [Phe-Pro-Val]₂-OC | Tyr-Pro-Val-OC-Val-Tyr-Leu |
| [Boc-Tle]₂-OC | [Phe-Ser-Val]₂-OC | [Lys-Lys-Gly-Gly]₂-OC |
| | | [SEQ ID NO: 35] |
| [Boc-Val]₂-OC | [Phe-Thr-Val]₂-OC | [Asp₂-Lys(Asp₂)]₂-OC |
| | | [SEQ ID NO: 29] |
| [Glu]₂-OC | [Phe-Tyr-Val]₂-OC | [Glu₂-Lys(Glu₂)]₂-OC |
| | | [SEQ ID NO: 30] |
| [Ile]₂-OC | [Pro-Pro-Cha]₂-OC | [Gly₂-Lys(-Gly₂)]₂-OC |
| | | [SEQ ID NO: 36] |
| [Leu]₂-OC | [Pro-Pro-Ile]₂-OC | [Phe₂-Lys(Phe₂)]₂-OC |
| | | [SEQ ID NO: 31] |
| [Lys]₂-OC | [Pro-Pro-Nle]₂-OC | [Pro₂-Lys(Pro₂)]₂-OC |
| | | [SEQ ID NO: 32] |
| [Phe]₂-OC | [Pro-Pro-Phe]₂-OC | [Tyr₂-Lys(Tyr₂)]₂-OC |
| | | [SEQ ID NO: 33] |
| [β-Ala]₂-OC | [Leu-Asp-Val]₂-OC | |
| [Val]₂-OC | [Leu-Glu-Val]₂-OC | |
| Val-OC-Gly | [Pro-Pro-Leu]₂-OC | |
| [Asp-Asp-Cha]₂-OC | [Pro-Glu-Val]₂-OC | |
| [Asp-Asp-Nle]₂-OC | [Pro-Gly-Val]₂-OC | |
| [Asp-Asp-Phe]₂-OC | [Pro-Ile-Val]₂-OC | |
| [Asp-Asp-Val]₂-OC | [Pro-Lys-Val]₂-OC | |
| [Asp-d-Asp-Ile]₂-OC | [Pro-Phe-Val]₂-OC | |
| [Asp-Glu-Val]₂-OC | [Pro-Ser-Val]₂-OC | |
| [Asp-Gly-Val]₂-OC | [Pro-Thr-Val]₂-OC | |
| [Asp-Ile-Val]₂-OC | [Pro-Tyr-Val]₂-OC | |
| [Asp-Leu-Val]₂-OC | [Pro-Pro-Val]₂-OC | |

Oxycodone conjugates also include the OAc and OEt derivatives of the above conjugates (in the case of mono-conjugates).

Peptide conjugates were synthesized by the general method described below.

General Structure of Oxycodone Derivatives:

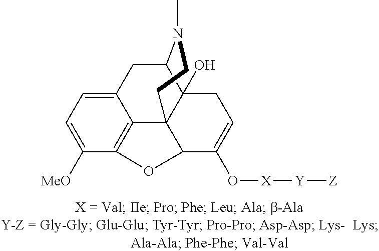

Synthetic Scheme of Oxycodone Derivatives:

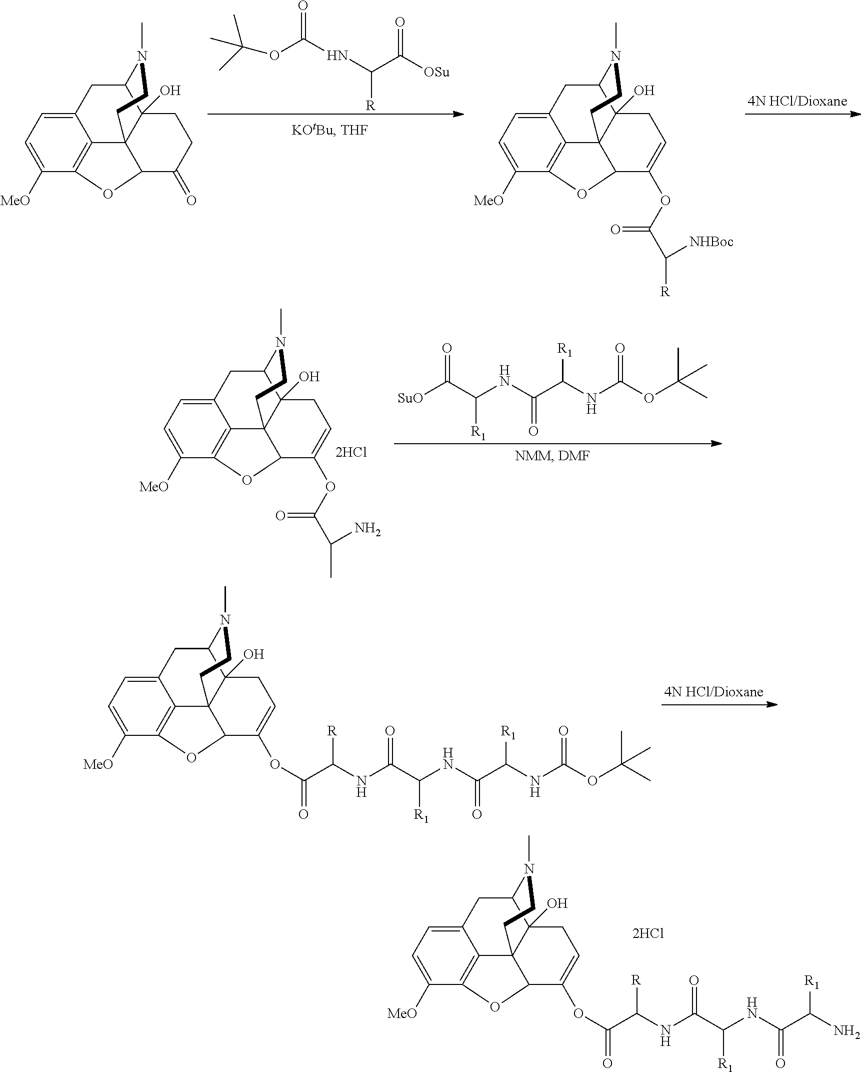

The above general synthesis scheme was applied to give the following preferred sequences of amino acids with oxycodone and bioavailability as set forth in Table 3.

TABLE 3
Exemplary bioavailability of oxycodone compounds.

| | | Oral |
|---|---|---|
| ENTRY | OXYCODONE COMPOUND | (% AUC) |
| | OXY | 100 |
| 1 | Gly-Gly-Val-OC | 123 |
| 2 | Ala-Ala-Val-OC | 85 |
| 3 | Glu-Glu-Val-OC | 55 |
| 4 | Lys-Lys-Val-OC | 108 |
| 5 | Leu-Leu-Val-OC | 81 |
| 6 | Tyr-Tyr-Val-OC | 124 |
| 7 | Pro-Pro-Val-OC | 152 |
| 8 | Phe-Phe-Val-OC | 32 |
| 9 | Asp-Asp-Val-OC | 40 |
| 10 | Val-Val-Val-OC | |
| *11 | Gly-Gly-Ile-OC | 224 |
| 12 | Glu-Glu-Ile-OC | 179 |
| 13 | Lys-Lys-Ile-OC | 74 |
| 14 | Tyr-Tyr-Ile-OC | 85 |
| 15 | Ile-Ile-Ile-OC | 83 |
| 16 | Pro-Pro-Ile-OC | 85 |
| 17 | Phe-Phe-Ile-OC | 59 |
| 18 | Glu-Glu-Pro-OC | 71 |
| 19 | Tyr-Tyr-Pro-OC | 59 |
| 20 | Gly-Gly-Phe-OC | 163 |
| 21 | Glu-Glu-Phe-OC | 49 |
| 22 | Lys-Lys-Phe-OC | 37 |
| 23 | Val-Val-Phe-OC | 120 |
| 24 | Tyr-Tyr-Phe-OC | 73 |
| 25 | Gly-Gly-Leu-OC | |
| 26 | Glu-Glu-Leu-OC | 80 |
| 27 | Val-Val-Leu-OC | |
| 28 | Lys-Lys-Leu-OC | 46 |
| 29 | Leu-Leu-Leu-OC | |
| 30 | Tyr-Tyr-Leu-OC | |
| 31 | Pro-Pro-Leu-OC | |
| 32 | Phe-Phe-Leu-OC | |
| 33 | Glu-Glu-Ala-OC | |
| 34 | Leu-Leu-Ala-OC | |
| 35 | Lys-Lys-Ala-OC | |
| 36 | Pro-Pro-Ala-OC | |
| 37 | Tyr-Tyr-Ala-OC | |
| 38 | Gly-Gly-β-Ala-OC | |
| 39 | Glu-Glu-β-Ala-OC | |
| 40 | Leu-Leu-β-Ala-OC | |
| 41 | Lys-Lys-β-Ala-OC | |
| 42 | Tyr-Tyr-β-Ala-OC | |
| 43 | OC-Succcinate | 79 |
| 44 | OC-β-alanine | 144 |

*Dosed at 40% higher levels than calculated each experiment conducted on n = 4 animals analysis by LC-MS

An iterative approach can be used to identify favorable conjugates by synthesizing and testing single amino acid conjugates, and then extending the peptide one amino acid at a time or through the attachment of peptides to yield dipeptide and tripeptide conjugates, etc. The parent single amino acid prodrug candidate may exhibit more or less desirable characteristics than its di- or tripeptide offspring candidates.

### I. Mono-Substituted Oxycodone Conjugates

### Single Amino Acids

### Example 1

### Phe-Oxycodone-Substitution at the 6 Position

To a solution of oxycodone-freebase (1.0 eq) in tetrahydrofuran (THF) (10 ml/mmol) was added K-O-t-butoxide (1.1 eq) or LiN(TMS)₂ (1.1 eq). After 5 minutes, Boc-Phe-OSu (1.1 eq) was added. The reaction was stirred at ambient temperatures for 18 hours, quenched with NH₄Cl, diluted with EtOAc, and solvents removed. Crude protected product was purified using chromatography. Deprotection occurred with 4N HCl in dioxane (20 ml/mmol) to obtain Phe-Oxycodone.

The following conjugates may be produced according to the above method:

Example: β-Leu-OC.

### Tripeptides

### Example 2

### General Synthesis of Mono-Substituted Tripeptide Oxycodone Conjugates Boc-Z—Y—X—O⁶-Oxycodone

To a solution of X—O⁶-Oxycodone.2HCl (1 mmol) in DMF were added NMM (10 mmol) and Boc-Z—Y—OSu (1.2 mmol). The reaction mixture was stirred at room temperature overnight. Solvent was evaporated to the residue was added saturated NaHCO₃ solution and stirred for 1 h. The precipitate was filtered, thoroughly washed with water and dried to give the title compound.

### Deprotection of Boc-Z—Y—X—O⁶-Oxycodone:

Deprotection is performed in the same manner as the general method mentioned above to give Z—Y—X—O⁶-Oxycodone.2HCl.

The following tripeptide conjugates may be produced according to the above method:

Examples: Phe-Tyr-Val-OC
Leu-Tyr-Val-OC

### II. Disubstituted Oxycodone Conjugates

### Disubstituted Single Amino Acid Oxycodone Conjugates

### Example 3

### General Synthesis of Disubstituted Oxycodone Conjugates Containing Identical Amino Acid: [Boc-X]₂-Oxycodone

To a solution of oxycodone free base (2.04 g, 6.47 mmol) in THF (˜35 ml) was added LiN(TMS)₂ (19.41 ml, 19.41 mmol) and stirred for ˜30 mins. To this was added solid Boc-X—OSu (X=amino acid, 21 mmol) at one time and the reaction mixture was stirred at room temperature overnight. The solution was neutralized with 1N HCl and the THF was removed under reduced pressure. The residue was diluted with EtOAc (200 mL), satd. NaHCO₃ (150 mL) was added and stirred for 1 h. EtOAc part was washed with NaHCO₃ and brine. Dried over Na₂SO₄ and evaporated to dryness. Compound was obtained by purification over silica gel column (30% EtOAc/Hexane).

### Deprotection of [Boc-X]₂-Oxycodone:

General method of deprotection: The above compound was reacted with 4N HCl/dioxane (25 mL/gm) at room temperature for 4 h. Solvent was evaporated and dried over vacuum to give X₂-Oxycodone.3HCl.

### Example 4

### General Synthesis of Disubstituted Oxycodone Conjugates Containing Different Amino Acids: Boc-X—O⁶-Oxycodone-O¹⁴—Y-Cbz

To a solution of Boc-X-Oxycodone (immol) in THF (10 mL) was added LiN(TMS)₂ (1.1 mmol) at 0° C. and the solution was stirred for 30 mins then Cbz-Y—OSu (1.25 mmol) was added. The reaction mixture was stirred at room temperature overnight. The solution was cooled down to 0° C., neutralized with 1N HCl and the organic part was evaporated. To the residue were added EtOAc (50 mL) and satd. NaHCO₃ (50 ml), stirred for 1 h. The organic part was washed with water, brine, dried over Na₂SO₄ and evaporated to dryness. The residue was purified over silica gel to give the title compound.

### Deprotection of Boc-X—O⁶-Oxycodone-O¹⁴—Y-Cbz.2HCl:

Boc-X—O⁶-Oxycodone-O¹⁴—Y-Cbz was deprotected following the general method for deprotection mentioned above to give X—O⁶-Oxycodone-O¹⁴—Y-Cbz.2HCl.

### Disubstituted Tripeptide Oxycodone Conjugates

### Example 5

### Synthesis of Tripeptide-OC-Tripeptide Conjugates Containing Two Tripeptides Each Individually Having Identical Amino Acid Sequences

### Synthesis of [Boc-Val]₂-OC:

To a solution of OC (2.04 g, 6.47 mmol) in tetrahydrofuran (THF) (˜35 ml) was added LiN(TMS)₂ (19.41 ml, 19.41 mmol) and stirred for ˜30 mins. To this was added solid Boc-Val-OSu (6.72 g, 21 mmol) at one time and the reaction mixture was stirred at room temperature overnight. The solution was neutralized with 1N HCl and the THF was removed under reduced pressure. The residue was diluted with ethyl acetate (EtOAc) (200 mL), satd. NaHCO₃ (150 mL) was added and stirred for 1 h. EtOAc part was washed with NaHCO₃ and brine. Dried over Na₂SO₄ and evaporated to dryness. Crude product was purified with either silica gel column. (30% EtOAc/Hexane).

Deprotection: For the deprotection of 2.5 g of [Boc-Val]₂-OC, 75-80 mL of 4N HCl/dioxane was used. Reaction was complete within 3-4 hours. Evaporate dioxane and dry over vacuum.

Coupling: To a solution of Val₂-OC.3HCl (250 mg, 0.4 mmol) in DMF (10-12 ml) were added NMM (10-12 eqv) and Boc-X—Y—OSu (2.6 eqv). The reaction mixture was stirred at RT overnight. Solvents were evaporated under reduced pressure. To the residue was added satd. NaHCO₃ (−30 mL) and stirred for 1 h. The white/pale yellow residue was filtered, thoroughly washed with water and dried in the vacuum oven at RT.

Deprotection: Deprotection was same as above method. For 100-200 mg of tripeptide derivative 10-15 ml 4N HCl/dioxane was used.

Deprotection of tripeptide derivatives containing Threonine and Serine: Tripeptide derivatives were dissolved in 95% TFA (5% water) and stirred for 4 h at room temperature. Solvent was evaporated and the residue was co-evaporated with toluene twice and dried over vacuum. 4N HCl/dioxane was added and stirred overnight. Product was evaporated to dryness and dried over vacuum.

### Example 6

### Synthesis of Tripeptide-OC-Tripeptide Conjugates Containing Two Tripeptides Each Individually Having Different Amino Acid Sequences

### Synthesis of [Boc-Z—Y—X]₂-Oxycodone [X, Y and Z are Amino Acids]

To a solution of X₂-Oxycodone 3HCl (1 mmol) in DMF (15-20 mL) were added NMM (10-12 eqv) and Boc-Z—Y—OSu (2.6 eqv). The reaction mixture was stirred at RT overnight. Solvent was evaporated under reduced pressure. To the residue was added satd. NaHCO₃ (−30 mL) and stir for 1-2 h. The white/pale yellow residue was filtered, thoroughly washed with water and dried in the vacuum oven at room temperature.

### Deprotection of [Boc-X—Y—Z]₂-Oxycodone:

Deprotection is same as general method mentioned above. For 100-200 mg of tripeptide derivative 10-15 ml 4N HCl/dioxane is used. Deprotection is done overnight to give [X—Y—Z]₂-Oxycodone.3HCl.

### Deprotection of Tripeptide Derivatives Containing Threonine and Serine:

First the tripeptide derivatives are dissolved 95% TFA (5% water) and stirred for 4 h at room temperature. Solvent is evaporated, the residue is co-evaporated with toluene twice and dried over vacuum. 4N HCl/dioxane is added and stirred overnight. Residue was evaporated to dryness and dried over vacuum.

### Synthesis of Boc-A-B—X—O⁶-Oxycodone-O¹⁴—Y—B-A-Boc (A,B,X,Y=Amino Acids):

To a solution of X—O⁶-Oxycodone-O¹⁴—Y.3HCl (1 mmol) and NMM (10 mmol) in DMF (10 mL) was added Boc-A-B—OSu (2.5 mmol) and the reaction mixture was stirred at room temperature overnight. Solvent was evaporated under reduced pressure and to the residue satd. NaHCO₃ (15 mL) was added and stirred for 1 h. The precipitate was filtered off and the residue was washed thoroughly with water and dried.

### Deprotection of Boc-A-B—X—O⁶-Oxycodone-O¹⁴—Y—B-A-Boc:

Deprotection is same as general method mentioned above. Deprotection is done overnight to give A-B—X—O⁶-Oxycodone-O¹⁴—Y—B-A.3HCl.

Synthesis of Boc-A-B—X—O⁶-Oxycodone-O¹⁴—Y—C-D-Boc=amino acids):

To a solution of Boc-A-B—X—O⁶-Oxycodone-O¹⁴—Y—NH₂ (1 mmol) in DMF (10 mL) were added NMM (5 mmol) and Boc-D-C—OSu (1.1 mmol) and the reaction mixture was stirred at room temperature overnight. Solvent was evaporated under reduced pressure and to the residue satd. NaHCO₃ was added and stirred for 1 h. The white precipitate was filtered, washed with water and dried.

### Deprotection of Boc-A-B—X—O⁶-Oxycodone-O¹⁴—Y—C-D-Boc:

Deprotection is same as general method mentioned above. Deprotection is done overnight to give A-B—X—O⁶-Oxycodone-O¹⁴—Y—C-D.3HCl.

### Disubstituted Tripeptide-Oxycodone-Single Amino Acid Conjugates

### Example 7

### Synthesis of Tripeptide-OC-Single Amino Acid Conjugates Containing a Tripeptide Having a Different Amino Acid Sequence

### Synthesis of Boc-A-B—X—O⁶-Oxycodone-O¹⁴—Y-Cbz:

To a solution of X—O⁶-Oxycodone-O¹⁴—Y-Cbz.2HCl (1 mmol) and NMM (10 mmol) in DMF (10 mL) was added Boc-A-B—OSu (1.1 mmol) and the reaction mixture was stirred at room temperature overnight. Solvent was evaporated under reduced pressure and to the residue satd. NaHCO₃ (20 mL) was added and stirred vigorously for 2-3 h. The precipitate was filtered off and the residue was washed thoroughly with water and dried.

### Synthesis of Boc-A-B—X—O⁶-Oxycodone-O¹⁴—Y—NH₂:

To a suspension of Boc-A-B—X—O⁶-Oxycodone-O¹⁴—Y-Cbz and Pd/C (25 Wt %) in EtOH (20 ml/gm) and cyclohexene (10 ml/gm) was heated under reflux for 30 mins. The reaction mixture was cooled down to room temperature and filtered. The filtrate was evaporated to dryness to give the title compound.

### Disubstituted Pentapeptide Oxycodone Conjugates

### Example 8

### Synthesis of Pentapeptide-OC-Pentapeptide Conjugates Containing Two Pentapeptides Each Having Different Amino Acid Sequences

### Synthesis of [Gly₂-Lys(-Gly₂)[SEQ ID NO: 36]]₂-Oxycodone

To a solution of (Gly)₂-Oxycodone (1.0 eq) in dimethylformamide (1 ml/mmol) was added 4-methylmorpholine (5.5 eq) followed by Boc-Gly₂-Lys-Gly-OSu [SEQ ID NO: 37] (4.1). Reaction was stirred at ambient temperature for 24 hours. Solvents were removed and crude product was purified by reverse phase HPLC, followed by HCl deprotection gave the title compound.

### [(I)-Lys-(d)-Lys-Leu]₂-Oxycodone

To a solution of (Leu)₂-Oxycodone (1.0 eq) in dimethylformamide (1 ml/mmol) was added 4-methylmorpholine (10 eq) followed by Boc-(1)-Lys(Boc)-(d)-Lys(Boc)-OSu (3 eq). Reaction was stirred at ambient temperature for 24 hours. Solvents were removed and crude product was purified by reverse phase HPLC.

### Bioavailability Studies of Oxycodone Conjugates

The invention is illustrated by pharmacokinetic studies with oxycodone that has been covalently modified by attachment to various moieties such as an individual amino acid, specific short chained amino acid sequences such as di-, tri-, and pentapeptides, or carbohydrates such as ribose, etc. Studies include pharmacokinetic evaluations of the various drug conjugates administered by the oral, intranasal, and intravenous routes. Collectively the compounds demonstrate that active agents may be modified by covalent attachment to various moieties and retain their therapeutic value at normal doses while preventing potential overdose by oral administration and prevention of abuse through intranasal and intravenous administration.

The Examples illustrate the applicability of attaching various moieties to oxycodone to reduce the potential for overdose while maintaining therapeutic value. The invention is illustrated by pharmacokinetic studies with various peptide opioid conjugates. The Examples illustrate the compounds and compositions for reducing the potential for overdose and abuse while maintaining therapeutic value wherein the active agent oxycodone (OC) is covalently attached to a chemical moiety. The compound which is di-substituted at the 6 and 14 position of oxycodone is termed [PPL]₂-OC.

Oral, intranasal, and intravenous bioavailability studies of oxycodone and oxycodone conjugates were conducted in male Sprague-Dawley rats. Doses of oxycodone hydrochloride and oxycodone conjugates containing equivalent amounts of oxycodone were administered in deionized water. Oral administration was in 0.5 ml by gavage needle. Intranasal doses were administered by placing 20 microliters into the nasal flares of rats anesthetized with isoflurane. Intravenous administration was in 0.1 ml by tail vein injection. Plasma was collected by retroorbital sinus puncture under isoflurane anesthesia. Oxycodone and oxymorphone (major active metabolite) concentrations were determined by LC/MS/MS.

### Example 9

### Decreased Oral Cₘₐₓ of Oxycodone Conjugates

Male Sprague-Dawley rats were provided water ad libitum, fasted overnight and dosed by oral gavage with oxycodone conjugates or oxycodone HCl. All doses contained equivalent amounts of oxycodone base. Plasma oxycodone concentrations were measured by ELISA (Oxymorphone, 102919, Neogen, Corporation, Lexington, Ky.) and/or LC/MS. The assay is specific for oxymorphone (the major oxycodone metabolite) and oxycodone. These examples illustrate that doses of oxycodone conjugates decrease the peak level (Cₘₐₓ) of oxycodone plus oxymorphone as compared to that produced by equimolar (oxycodone base) doses of oxycodone HCl when given by the oral route of administration.

### Example 10

### Oral Bioavailability of a Peptide-Oxycodone Conjugates at a Dose (2.5 mg/kg) Approximating a Therapeutic Human Dose

This example illustrates that when the peptide PPL is conjugated (disubstituted at the 6 and 14 positions) to the active agent oxycodone oral bioavailability is maintained as compared to an equimolar oxycodone dose when the dose administered is 1 mg/kg. This dose is the equivalent of a human dose of 25 to 35 mg for an individual weighing 70 kg (148 lbs) according to Chou et al.

TABLE 4
Oral Pharmacokinetics of Oxycodone vs. [PPL]₂-OC (2.5 mg/kg dose).

| | Hours | | | | | AUC (ng/ml h) | Percent | Cmax | Percent |
|---|---|---|---|---|---|---|---|---|---|
| Drug | 0.5 | 1.5 | 3 | 5 | 8 | 0-8 h | OC | ng/ml | OC |
| Oxycodone Bitartrate | 145 | 27 | 11 | 2 | 1 | 168 | 100 | 145 | 100 |
| [PPL]₂-OC | 124 | 78 | 46 | 1 | 3 | 278 | 165 | 124 | 86 |

oxycodone plus oxymorphone

### Example 11

### Bioavailability of [PPL]₂-Oxycodone by the Intranasal Route

This example illustrates that when [PPL]₂ is conjugated to the active agent oxycodone the bioavailability by the intranasal route is substantially decreased thereby diminishing the possibility of overdose.

### Example 12

### Bioavailability of [PPL]₂-Oxycodone by the Intravenous Route

This example illustrates that when [PPL]₂ is conjugated to the active agent oxycodone the bioavailability by the intravenous route is substantially decreased thereby diminishing the possibility of overdose.

### Summary of In Vivo Testing of Abuse Resistant Oxycodone Conjugates.

In vivo testing of oxycodone conjugates demonstrates for instance decreased oral Cₘₐₓ, decreased intranasal bioavailability (AUC and Cₘₐₓ), and decreased intravenous bioavailability (AUC and Cₘₐₓ) and is described in further detail below.

### Example 13

### Decreased Intranasal Bioavailability (AUC and Cₘₐₓ) of Oxycodone Conjugates

Male Sprague-Dawley rats were provided water ad libitum and doses were administered by placing 0.02 ml of water containing oxycodone conjugates or oxycodone bitartrate into the nasal flares. All doses contained equivalent amounts of oxycodone base. Plasma oxycodone concentrations were measured by ELISA (Oxymorphone, 102919, Neogen, Corporation, Lexington, Ky.) and/or LC/MS. The assay is specific for oxymorphone (the major oxycodone metabolite) and oxycodone. These examples illustrate that oxycodone conjugates decrease the peak level (Cₘₐₓ) and total absorption (AUC) of oxycodone plus oxymorphone as compared to those produced by equimolar (oxycodone base) doses of oxycodone HCl when given by the intranasal route of administration.

### Example 14

### Decreased Intravenous Bioavailability (AUC and Cₘₐₓ) of Oxycodone Conjugates

Male Sprague-Dawley rats were provided water ad libitum and doses were administered by intravenous tail vein injection of 0.1 ml of water containing oxycodone conjugates or oxycodone HCl. All doses contained equivalent amounts of oxycodone base. Plasma oxycodone concentrations were measured by ELISA (Oxymorphone, 102919, Neogen, Corporation, Lexington, Ky.) and/or LC/MS. The assay is specific for oxymorphone (the major oxycodone metabolite) and oxycodone. This example illustrates that an oxycodone conjugate decreases the peak level (Cₘₐₓ) and total absorption (AUC) of oxycodone plus oxymorphone as compared to those produced by an equimolar (oxycodone base) dose of oxycodone HCl when given by the intravenous route of administration.

Additional bioavailability date is provided in Tables 5-7 for some exemplary compounds.

TABLE 5
Oxycodone Compounds (Class/Oral Bioavailability)

| Formula | Class | AUC % |
|---|---|---|
| NA | NA | 100 |
| (SGV)₂-OC | Disubstituted peptide | 103 |
| (EDV)₂-OC | Disubstituted peptide | 73 |
| (VEV)₂-OC | Disubstituted peptide | 104 |
| YYV-OC | Monosubstituted peptide | 124 |
| PPV-OC | Monosubstituted peptide | 152 |
| PPI-OC | Monosubstituted peptide | 85 |
| OC-β-Alanine | Monosubstituted Single | 144 |
| | Non-natural Amino Acid | |

TABLE 6
Intranasal Bioavailability of Oxycodone Compounds

| Formula | Class | AUC % |
|---|---|---|
| NA | NA | 100 |
| (SGV)₂-OC | Disubstituted peptide | 64 |
| (EDV)₂-OC | Disubstituted peptide | 20 |
| (VEV)₂-OC | Disubstituted peptide | 39 |

TABLE 7
Intravenous Bioavailability of Oxycodone Compounds

| Formula | Class | AUC % |
|---|---|---|
| NA | NA | 100 |
| (SGV)₂-OC | Disubstituted peptide | 52 |

Collectively, the examples illustrate the application of the invention for reducing the overdose potential of narcotic analgesics. These examples establish that an active agent can be covalently modified by attachment of a chemical moiety in a manner that maintains therapeutic value over a normal dosing range, while substantially decreasing if not eliminating the possibility of overdose by oral, intranasal, or intravenous routes of administration with the active agent.

1-23. (canceled)

24. A composition comprising an oxycodone covalently bound to at least one chemical moiety through a ketone, hydroxyl, or both in a pharmaceutically acceptable oral dosage form.

25. The composition of claim 24, wherein said chemical moiety is a polypeptide of two or more amino acids.

26. The composition of claim 25, wherein the amino acid adjacent to the said oxycodone is a nonpolar amino acid.

27. The composition of claim 26, wherein the amino acid adjacent to the said oxycodone is a branched nonpolar amino acid.

28. The composition of claim 27, wherein the amino acid adjacent to the said oxycodone is a branched nonpolar amino acid chosen from the naturally occurring amino acids valine, leucine, or isoleucine.

29. The composition of claim 25, wherein said polypeptide is comprised of one or more of the naturally occurring amino acids.

30. The composition of claim 25, wherein said chemical moiety is comprised of one or more of the D-isomers of the naturally occurring amino acids.

31. The composition of claim 25, wherein said chemical moiety is comprised of one or more of the L-isomers of the naturally occurring amino acids.

32. The composition of claim 25, wherein said chemical moiety is comprised of a mixture of one or more of the D- or L-isomers of the naturally occurring amino acids.

33-35. (canceled)

36. The composition of claim 25, wherein said polypeptide comprises the amino acid sequence selected from the group Pro-Pro-Leu, Pro-Pro-Ile, Val-Val, Lys-Lys, Gly-Gly-Ile, Phe-Phe-Ile, Phe-Phe-Leu, Thr-Thr-Val, Tyr-Tyr-Val, Tyr-Tyr-Phe, Glu-Glu-Val, Asp-Asp-Val, Lys-Lys-Val, Glu-Glu-Phe-Phe-Ile (SEQ ID NO: 34), Glu-Glu-Phe-Phe-Phe [SEQ ID NO: 151, Tyr-Tyr-Ile, Asp-Asp-Ile, Tyr-Tyr-Phe-Phe-Phe-Ile (SEQ ID NO: 27], Tyr-Tyr-Lys-Tyr-Tyr [SEQ ID NO: 33], Phe-Phe-Lys-Phe-Phe [SEQ ID NO: 31], Glu-Glu-Phe-Phe-Leu (SEQ ID NO: 10], {Lys-Lys-Gly-Gly (SEQ ID NO: 35), and [(1)-Lys-(d)-Lys-Leu]₂.

37. The composition of claim 25, wherein the dosage form is a tablet, a capsule, oral solution, an oral suspension, or a controlled release formulation.

38. (canceled)

39. The composition of claim 24, wherein said chemical moiety is a polypeptide of fewer than eight amino acids.

40-85. (canceled)

86. A compound comprising oxycodone covalently attached to one or more peptide.

87. The compound of claim 86 comprising oxycodone covalently attached to one or more peptide comprising the amino acid sequence selected from the group consisting of Pro-Pro-Ile. Phe-Phe-Lys-Phe-Phe [SEQ ID NO: 31], Tyr-Tyr-Lys-Tyr-Tyr [SEQ ID NO: 33], Tyr-Tyr-Phe, Phe-Phe-Leu, Phe-Phe-Val, Phe-Phe-Ile, Glu-Glu-Phe-Phe-Phe-Ile [SEQ ID NO: 34]. Tyr-Tyr-Ile, Lys-Lys-Val, Asp-Asp-Val, Tyr-Tyr-Val, Pro-Pro-Val, Glu-Glu-Val, and Thr-Thr-Val.

88. The compound of claim 87, wherein said one or more peptide comprises the amino acid sequence Pro-Pro-Ile.

89. A composition comprising a compound of claim 86.

90-93. (canceled)

94. A compound comprising oxycodone covalently bound to a single amino acid a dipeptide, a tripeptide, a tetrapeptide, or a pentapeptide.

95-99. (canceled)

100. A compound selected from:
(Bio-Gly₂-Ile)₂-oxycodone,
(Bio-Gly₂-Leu)₂-oxycodone,
(Gal-Gly₂-Ile)₂-oxycodone
(Gal-Gly₂-Leu)₂-oxycodone,
(Gal-Prθ2-Ile)₂-oxycodone,
(Gal-Prθ2-Leu)₂-oxycodone,
(Glu)₂-oxycodone,
(Glu-Asp-Val)₂-oxycodone,
(Gly-Leu-Val)₂-oxycodone,
(Ile)₂-oxycodone,
(Ile-Tyr-Val)₂-oxycodone,
(Ile-Tyr-Val)-oxycodone-(Gly-Tyr-Ile),
(He-Tyr-Val)-oxycodone-(Val-Glu-Gly),
(Leu)₂-oxycodone,
(Leu-Tyr-Val)-oxycodone-(Gly-Tyr-Leu),
(Leu-Tyr-Val)-oxycodone-(Val-Glu-Gly),
(Lvs)₂-oxycodone,
(Lys-Lys-Gly-Gly)₂-oxycodone [SEQ ID NO: 35],
(Lys-Ser-Val)₂-oxycodone,
(Nia-Gly₉-Ile)₂-oxycodone,
(Nia-Gly₂-Leu)₂-oxycodone,
(Phe)₂-oxycodone,
(Phe-Val-Val)₂-oxycodone,
(Ser-Thr-Val)₂-oxycodone,
(Tyr-Pro-Val)₂-oxycodone,
(Val)₂-oxycodone,
[(1)-Lys-(d)-Lys-Leu]₂-oxycodone,
[Glu-Pro-Val]₂-oxycodone,
[Glu-Tyr-Val]₂-oxycodone,
[Gly₂-Lys(-Gly₂)]2-oxycodone [SEQ ID NO: 36],
[Gly-Glu-Val]₂-oxycodone,
[Gly-Tyr-Valj₂-oxycodone,
[Ile-Tyr-Val]₂-oxycodone,
[Leu-Tyr-Val]₂-oxycodone,
[Pro-Glu-Val]₂-oxycodone,
[Ser-Gly-Val]₂-oxycodone,
[Tyr-Tyr-Val]₂-oxycodone,
[Val-Glu-Val]₂-oxycodone,
Ala-Ala-Val-oxycodone,
Asp-Asp-Ile-oxycodone,
Asp-Asp-Val-oxycodone,
Glu-Glu-Ala-oxycodone,
Glu-Glu-Ile-oxycodone,
Glu-Glu-Leu-oxycodone,
Glu-Glu-Phe-oxycodone,
Glu-Glu-Phe-Phe-Ile-oxycodone [SEQ ID NO: 34],
Glu-Glu-Phe-Phe-Phe-oxycodone [SEQ ID NO: 15],
Glu-Glu-Pro-oxycodone,
Glu-Glu-β-Ala-oxycodone,
Glu-Glu-Val-oxycodone,
Glu-Leu-Val-oxycodone,
Glu-Tyr-Val-oxycodone,
Gly-Gly-Ile-oxycodone,
Gly-Gly-Leu-oxycodone,
Gly-Gly-Phe-oxycodone,
Gly-Gly-β-Ala-oxycodone,
Gly-Gly-Val-oxycodone,
Ile-Ile-Ile-oxycodone,
Ile-oxycodone,
Ile-Tyr-Val-oxycodone,
Leu-Leu-Ala-oxycodone,
Leu-Leu-Leu-oxycodone,
Leu-Leu-Val-oxycodone,
Leu-Leu-β-Ala-oxycodone,
Leu-oxycodone,
Lys-Lys-Ala-oxycodone,
Lys-Lys-Ile-oxycodone,
Lys-Lys-Leu-oxycodone,
Lys-Lys-oxycodone,
Lys-Lys-Phe-oxycodone,
Lys-Lys-Val-oxycodone,
Lys-Lys-β-Ala-oxycodone,
oxycodone-succinate,
oxycodone-β-alanine,
Phe-oxycodone,
Phe-Phe-Ile-oxycodone,
Phe-Phe-Leu-oxycodone,
Phe-Phe-Lys-Phe-Phe-oxycodone [SEQ ID NO: 31],
Phe-Phe-Val-oxycodone,
Pro₂-Ile-oxycodone,
Pro₂-Leu-oxycodone,
Pro-Glu-Val-oxycodone,
Pro-Pro-Ala-oxycodone,
Pro-Pro-Ile-oxycodone,
Pro-Pro-Leu-oxycodone,
Pro-Pro-Val-oxycodone,
Thr-Thr-Val-oxycodone,
Tyr-Tyr-Ala-oxycodone,
Tyr-Tyr-Ile-oxycodone,
Tyr-Tyr-Leu-oxycodone,
Tyr-Tyr-Lys-Tyr-Tyr-oxycodone [SEQ ID NO: 33],
Tyr-Tyr-Phe-oxycodone,
Tyr-Tyr-Phe-Phe-Ile-oxycodone [SEQ ID NO: 27],
Tyr-Tyr-β-Ala-oxycodone,
Tyr-Tyr-Val-oxycodone,
Val-oxycodone,
Val-oxycodone-Gly,
Val-Val-Leu-oxycodone,
Val-Val-oxycodone,
Val-Val-Phe-oxycodone, or
Val-Val-Val-oxycodone.

101-208. (canceled)

209. A composition comprising the compound from claim 87.

210-221. (canceled)

222. A composition comprising the compound from claim 94.

223. A composition comprising the compound from claim 100.
